(19) United States
(12) Patent Application Publication
(10) Pub. No.: US 2024/0304275 A1
(43) Pub. Date: Sep. 12, 2024

(54) METHOD FOR DETERMINING EVOLUTIONARY PRIMITIVE ANCESTRY OF GOJI BERRY AND USE THEREOF

(71) Applicant: WOLFBERRY SCIENCE INSTITUTE, NAAFS, Yinchuan (CN)

(72) Inventors: Haijing YUAN, Yinchuan (CN); Yu CHEN, Yinchuan (CN); Haiyan YUAN, Yinchuan (CN); Lanying LIU, Yinchuan (CN); Chun'e ZHANG, Yinchuan (CN); Xiangcai LIU, Yinchuan (CN); Xu FENG, Yinchuan (CN); Liguo DONG, Yinchuan (CN); Mei TIAN, Yinchuan (CN); Jinzhong ZHU, Yinchuan (CN)

(73) Assignee: WOLFBERRY SCIENCE INSTITUTE, NAAFS, Yinchuan (CN)

(21) Appl. No.: 18/229,784

(22) Filed: Aug. 3, 2023

(30) Foreign Application Priority Data
Mar. 10, 2023 (CN) 202310233664.1

(51) Int. Cl. G16B 10/00; C12Q 1/6895; G06N 3/123; G16B 20/20; G16B 20/40; G16B 30/10

(52) U.S. Cl. CPC G16B 10/00; C12Q 1/6895; G06N 3/123; G16B 20/20; G16B 20/40; G16B 30/10; C12Q 2600/13; C12Q 2600/156

(57) ABSTRACT

Provided is a method for determining an evolutionary primitive ancestry of Goji berry and use thereof, which relate to the field of species origin and genetic evolution. The method for determining an evolutionary primitive ancestry of Goji berry includes developing genome-wide molecular markers of Goji berries based on Specific-Locus Amplified Fragment Sequencing (SLAF-seq) technology, and determining the primitive ancestry of Goji berry through bioinformatic analysis. The selected Goji berries include not only wild Goji berries from Ningxia, Gansu, Qinghai, Xinjiang, Shaanxi, Inner Mongolia and Henan, but also Korean and Mexican Goji berry germplasms, with a wide range of sample sources, strong representation and large amount of information. The original ancestral genetic taxa of each Goji berry can be accurately determined through big data analysis. The gap in the origin and genetic evolution of Goji berry is thus fulfilled. The method has a good prospect in application.

### CROSS-REFERENCE TO RELATED APPLICATION

This patent application claims the benefit and priority of Chinese Patent Application No. 2023102336641, entitled “METHOD FOR DETERMINING EVOLUTIONARY PRIMITIVE ANCESTRY OF GOJI BERRY AND USE THEREOF” filed on Mar. 10, 2023, the disclosure of which is incorporated by reference herein in its entirety as part of the present application.

### TECHNICAL FIELD

The present disclosure relates to the technical field of species origin and genetic evolution, in particular to a method for determining the evolutionary primitive ancestry of Goji berry and use thereof.

### BACKGROUND ART

Goji berry, a deciduous shrub plant of genus Lycium, Solanaceae family, is a traditional and valuable Chinese herbal medicine. Records about the medicinal use and cultivation of Goji berry could trace back to as early as in “Shen Nong's herbal classic” and “Compendium of Materia Medica”. There are about 80 species of the genus worldwide, with a discrete global distribution, ranging from South America and North America to Australia, Eurasia, the Pacific Islands and South Africa. Symon suggests that this discrete distribution is probably due to the breakup and drift of Gondwana (Hawks J G, Lester R N, Nee M, et al. Solanaceae II: taxonomy-chemistry-evolution [M]. London: Kew Publishing, 1991: 1139). Some researchers also believe that it is due to the spread of the species of the genus itself.

At present, the origin of Lycium species in the world academic community has been inconclusive. There are a variety of theories, such as “Lycium species originated in the American continent”, “Lycium species originated in South Africa”, “Lycium species originated in China”, and so on. The general preference is for the “American continent origin” theory. These theories are based on the investigation of the botanical traits of Lycium, and the requirements for samples are not clear enough, resulting in a gap in the study on the original ancestry and genetic evolution of Lycium.

### SUMMARY

In view of this, it is an object of the present disclosure to provide a method for determining the evolutionary primitive ancestry of Goji berry. The method adopts a variety of samples from a wide range of sources, being with a large spatial, temporal and geographical span, which provides vast information, so that the evolutionary primitive ancestry of Goji berry are determined more accurately.

To solve the above technical problems, the present disclosure provides the following technical solutions:

The present disclosure provides a method for determining the evolutionary primitive ancestry of Goji berry, including steps of:
digesting DNA of a Goji berry sample with restriction endonucleases RsaI and HinCII, and subjecting digested fragments to high-throughput sequencing and bioinformatic analysis to obtain single nucleotide polymorphism (SNP) markers; and determining the evolutionary primitive ancestry of the Goji berry sample by genetic analysis of the SNP markers; where the Goji berry sample includes all species of Chinese Goji berry germplasms of 7 species and 3 varieties, germplasms of Korea in northeast Asia and germplasms of Mexico in America.

In some embodiments, a tree from which the Goji berry sample comes is from 3 to 156 years old and a sampling site is from 320 m to 3231 m in altitude.

In some embodiments, the digested fragments have a length of 364-414 bp.

In some embodiments, the digested fragments at a 3′ end, the digested fragments are subjected to A-tailing, ligation with adapters, PCR amplification, purification, mixing, and gel cutting to select target fragments, and high throughput sequencing is carried out after library quality control.

In some embodiments, the bioinformatic analysis includes acquisition of polymorphic Specific-Locus Amplified Fragment (SLAF) tags and acquisition of the SNP markers.

In an embodiment, the acquisition of polymorphic SLAF tags includes clustering reads from sequencing of different Goji berry samples based on sequence similarity.

In another embodiment, the acquisition of SNP markers includes steps of:
mapping the reads to a reference genome, with a sequence type that has the highest depth in each SLAF tag as a reference sequence, developing SNPs with two methods, GATK and samtools, respectively, and intersecting the SNPs obtained by the two methods to attain SNP markers.

In some embodiments, the genetic analysis includes phylogenetic tree analysis, population structure analysis, and principal component analysis (PCA) and linkage disequilibrium analysis.

The present disclosure also provides use of the method described above in determining a primitive ancestry of Goji berry or genetic evolution of Goji berry.

The present disclosure provides a method for determining an evolutionary primitive ancestry of Goji berry, including developing molecular markers for Goji berry material by Specific-Locus Amplified Fragment Sequencing (SLAF-seq) technology to obtain genome-wide molecular markers, and conducting bioinformatic analysis to determine the primitive ancestry of Goji berry. The selected Goji berry material not only includes wild Goji berry from Ningxia, Gansu, Qinghai, Xinjiang, Shaanxi, Inner Mongolia and Henan, but also includes 7 species and 3 variants of Chinese Goji berry germplasms, and Korean and Mexican Goji berry germplasms. The samples are of wide sources and strong representation, and contain a large amount of information. The original ancestral genetic taxa of each Goji berry can be accurately determined through big data analysis. The gap in the origin and genetic evolution of Goji berry is thus fulfilled. The method has a good prospect in application.

### BRIEF DESCRIPTION OF THE DRAWINGS

FIG. 1 shows a flow chart of the SLAF experiment.

FIGS. 2A-2J show the distribution of quality (Q) values in sequencing, where the x axis indicates base positions of the reads and the y axis indicates the Q scores of the single bases.

FIGS. 3A-3J show the distribution of base content, where the x axis indicates base positions of the reads and y axis indicates the proportion of bases.

FIG. 4 shows a bioinformatics analysis process.

FIG. 5 shows a flow chart of SLAF tag development.

FIG. 6 shows a phylogenetic tree of 110 Goji berry germplasms.

FIGS. 7A-7C show a 3D clustering plot by principal component analysis (PCA) of 110 samples of Goji berry germplasms.

FIGS. 8A-8C show two-dimensional clustering a plot by PCA of 110 samples of Goji berry germplasms.

FIG. 9 shows clustering of samples corresponding to each K value of Admixture.

FIG. 10 shows cross-validation error rates of each K value of Admixture.

### DETAILED DESCRIPTION OF THE EMBODIMENTS

The present disclosure provides a method for determining an evolutionary primitive ancestry of Goji berry, including steps of:
digesting DNA of a Goji berry sample with restriction endonucleases RsaI and HinCII, and subjecting digested fragments to high-throughput sequencing and bioinformatic analysis to obtain SNP markers; and determining the evolutionary primitive ancestry of the Goji berry sample by genetic analysis of the SNP markers; and
the Goji berry sample includes seven species and three varieties of Chinese Goji berry germplasms, germplasms from Korea in northeastern Asia and germplasms from Mexico in the Americas.

In the present disclosure, a sample size of the Goji berry sample is preferably at least 100, more preferably at least 110. In the present disclosure, seven species and three varieties of Chinese Goji berry germplasms preferably include Chinese Goji berry cultivars (currently not yet classified in the academic community of botany), wild Goji berry in northwest China (including Ningxia, Gansu, Qinghai, Xinjiang, Shaanxi, Inner Mongolia and other wild Goji berry producing regions); a tree from which the Goji berry sample comes is preferably 3 to 156 years old, and a sampling site is from 320 m to 3231 m in altitude. In the present disclosure, the Goji berry sample preferably has the following characteristics: (1) a wide range of sources: covering not only all species of Goji berry germplasms in China, but also germplasms from northeast Asia, Korea, Mexico, America; including not only recognized Goji berry species and varieties in China, but also China's wild Goji berry and cultivated Goji berry in the provinces of the northwest region, the main source of wild Goji berry species; (2) a large spatial and temporal span: covering not only Goji berry samples derived from trees with more than 100 years old, such as trees that in Ningxia, Inner Mongolia, where the oldest tree is 156 years old, as well as wild Goji berry trees from Wulonggou, Dagele Township, Dulan County, Qinghai Province, known as the origin of Goji berry “ancestral land”, but also wild Goji berry samples derived from trees of about 3 years old; (3) a large altitude, longitude and latitude span: covering samples spanning from 320 m in altitude in Jinghe County, Xinjiang to 3231 m in Wulonggou, Dagele Township, Dulan County, Qinghai. The Goji berry sample in the present disclosure has a large sample size, wide range of sources and representation, and thus is conductive to the determination of the original ancestral genetic taxa of Goji berry, facilitating the study of the origin and genetic evolution of Goji berry.

In the present disclosure, the DNA of the Goji berry sample is digested. In some embodiments of the present disclosure, the length of the digested fragments is 364-414 bp. In some embodiments of the present disclosure, the restriction endonucleases RsaI and HinCII and the length of the digested fragments are predicted by simulating restriction digestion on a reference genome of related species, according to the selection principle for digestion. The selection principle for digestion is preferably shown as follows: (1) the proportion of digested fragments located in repeated sequences should be as low as possible; (2) the digested fragments should be distributed as evenly as possible on the genome; (3) the length of the digested fragments should match the specific experimental system; (4) the final number of digested fragments (SLAF tags) obtained should meet the requirement for the tag number. In some embodiments of the present disclosure, the reference genome is a Capsicum frutescens genome. In the present disclosure, there is no special limitation on the method for DNA extraction of the Goji berry sample, and it is sufficient to use conventional DNA extraction methods in the field. In a specific embodiment of the present disclosure, the DNA of the Goji berry sample is extracted by the hexadecyltrimethylammonium bromide (CTAB) method.

In some embodiments of the present disclosure, the digested fragments are subjected to A-tailing at 3′ end, ligation with adapters PCR amplification, purification, mixing, and gel cutting to select target fragments, and high throughput sequencing is carried out after library quality control. Data obtained from the high-throughput sequencing is identified by Dual-index to obtain the reads of Goji berry samples, and after adaptor filtration of sequencing reads, evaluation of quality and data volume is performed. In some embodiments of the present disclosure, the platform for high-throughput sequencing is Illumina platform.

In some embodiments of the present disclosure, the bioinformatic analysis includes acquisition of polymorphic SLAF tags and acquisition of SNP markers. In the present disclosure, the acquisition of polymorphic SLAF tags include clustering reads from sequencing of different Goji berry samples based on sequence similarity; the reads clustered together are derived from one SLAF fragment (SLAF tag). The sequence similarity of the same SLAF tag between different samples is much higher than that between different SLAF tags; the SLAF tag with sequence differences (i.e., with polymorphism) between different samples is a polymorphic SLAF tag.

In the present disclosure, the acquisition of SNP markers includes steps of: mapping the reads to a reference genome, with a sequence type that has the highest depth in each SLAF tag as a reference sequence, developing SNPs with two methods, GATK and samtools, respectively, and intersecting the SNPs obtained by the two methods to attain SNP markers. In the present disclosure, the reference genome is Capsicum frutescens genome; and the software for mapping is Burrows-Wheeler Aligner (BWA).

In the present disclosure, the genetic analysis includes phylogenetic tree analysis, population structure analysis, principal component analysis and linkage disequilibrium analysis.

The present disclosure also provides use of the above method in determining a primitive ancestry of Goji berry or the genetic evolution of Goji berry.

In order to make the purpose, technical solutions and advantages of the present disclosure clearer and more understandable, the disclosure is described in detail below in conjunction with the examples, but they are not to be construed as limiting the protection scope of the disclosure.

In the following examples, if not otherwise specified, methods used are conventional methods.

The materials, reagents, etc. used in the following examples, if not otherwise specified, are available from commercial sources.

### Example 1

### 1. Material Collection and Taste Identification

A systematic survey method was used to collect 110 fresh leaf samples of Goji berry material from northwest China, including wild Goji berry from Ningxia, Gansu, Qinghai, Xinjiang, Shaanxi, Inner Mongolia and Henan, as well as Korean and Mexican Goji berry germplasms, 7 species and 3 varieties of Goji berry germplasms, and Chinese Goji berry cultivars, with GPS positioning and photography of wild Goji berry germplasms.

In view of the lack of instruments for rapid bitterness detection, a survey team of 3-5 researchers was formed to taste and determine the bitterness and sweetness of the mature fresh fruit. The bitterness of the mature fresh fruit of wild bitter Goji berry in Yuanhe Village, Xi'an Town, Haiyuan County, Ningxia Province was set at 10 degrees, the bitterness of mature fresh fruit of Ningqi 7 was set at 0 degree, and then 11 levels of bitterness and sweetness would be set in turn with reference to both, to observe the botanical traits of wild Goji berry germplasms. The results are shown in Table 4.

2. Extraction of DNA from Fresh Leaves of Goji Berry

Extraction of DNA was completed using CTAB method.

The DNA was extracted from 110 fresh leaves of Goji berry germplasms freezed with liquid nitrogen. The extraction was performed according to Doy le J J, Doy le J L, 1987. A rapid DNA isolation procedure for small quantities of fresh leaf tissue. Phytochem Bull, 19: 11˜15.

### 3. Digestion Protocol Design

The following principles were used to select the most suitable digestion protocol: (1) the proportion of digested fragments located in repeated sequences should be as low as possible; (2) the digested fragments should be distributed as evenly as possible on the genome; (3) the length of the digested fragments should match the specific experimental system; (4) the final number of digested fragments (SLAF tags) obtained should meet the requirement for the tag number.

Based on the above selection principles, the restriction endonuclease combination of RsaI+HinCII was determined, and fragments with a length of 364-414 bp were defined as SLAF tags.

### 4. Digestion Experiment

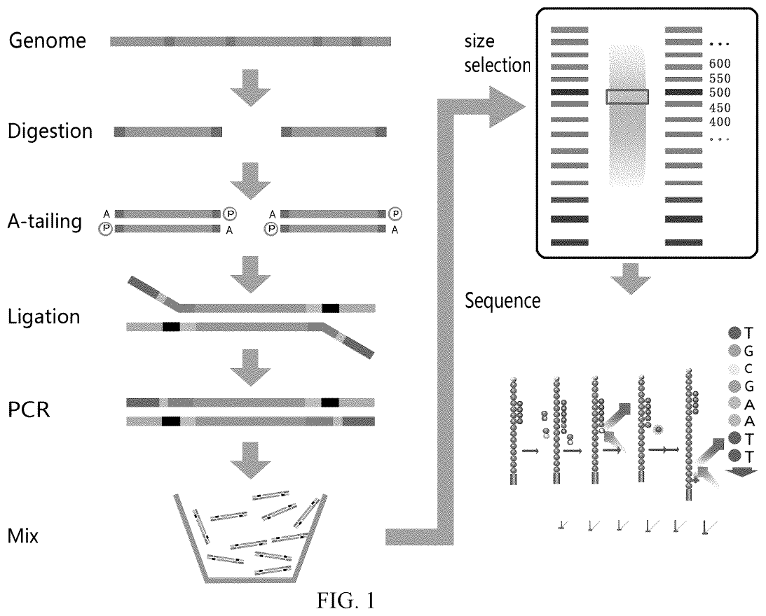

According to the digestion protocol selected in step 3, the genomic DNA of each qualified sample was digested separately. A single nucleotide adenine overhang was added to the digested fragments at the 3′ end, the digested fragments were ligated with Dual-index sequencing adaptors, and subjected to PCR amplification, purification, mixing, gel cutting to select the target fragments, and the target fragments were sequenced on Illumina platform after library quality control. The experimental flow is shown in FIG. 1.

### 5. Statistics and Evaluation of the Sequencing Data

The raw data obtained from sequencing were identified using Dual-index to obtain the reads of each sample, and after filtration of adaptors for the sequencing of the reads, the sequencing quality and data volume were evaluated. To ensure the analysis quality of the project, a read length of 126 bp×2 was used for the subsequent data evaluation and analysis data.

### (1) Sequencing Quality Value Distribution Check

Sequencing quality score Q is an important index for assessing calling error of single base in high-throughput sequencing, and a higher Q score corresponds to a lower probability of base calling error. The equation between the probability of base calling error P and Q-score is

$${Q}_{- score} = - 10 \times {log}_{10} P .$$

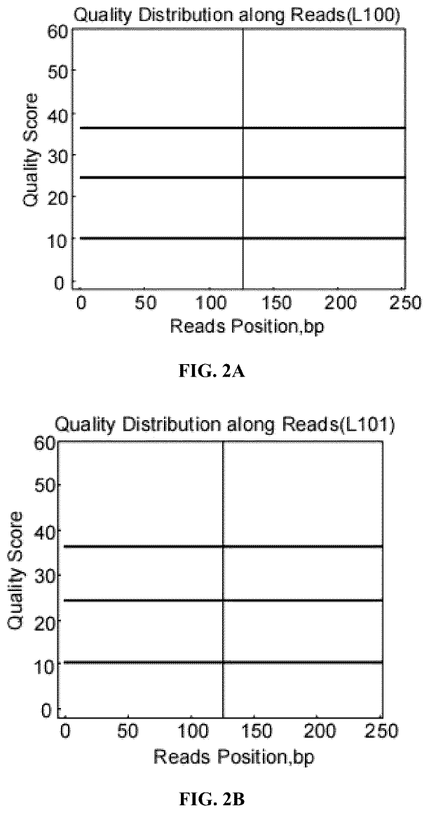

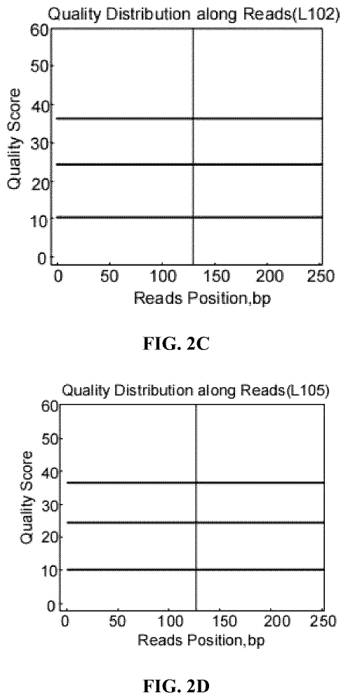

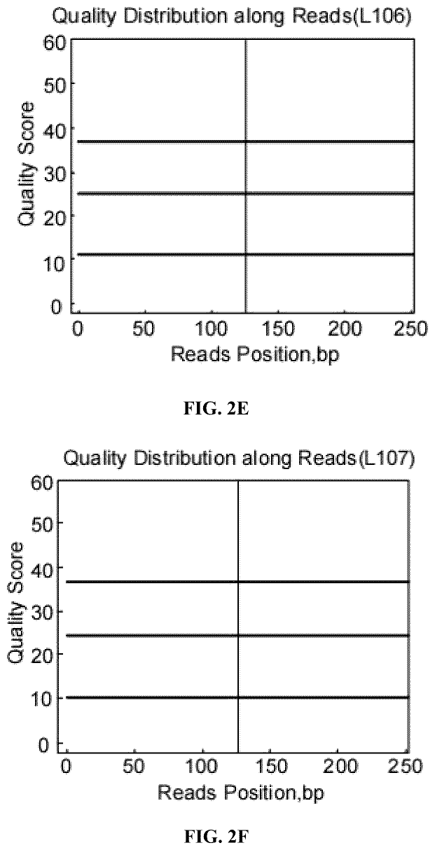

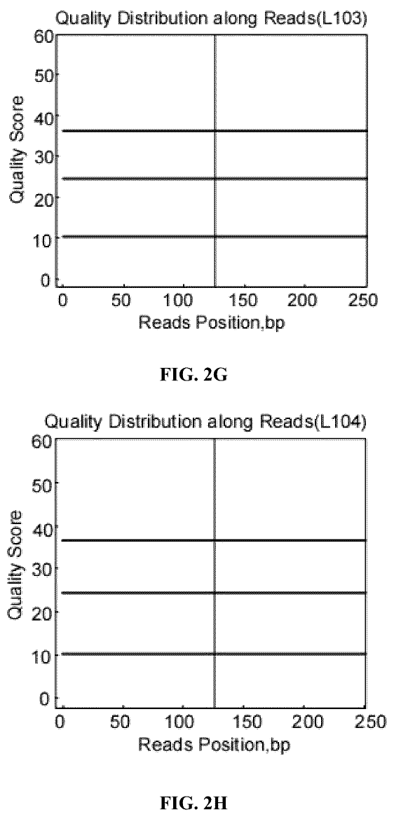

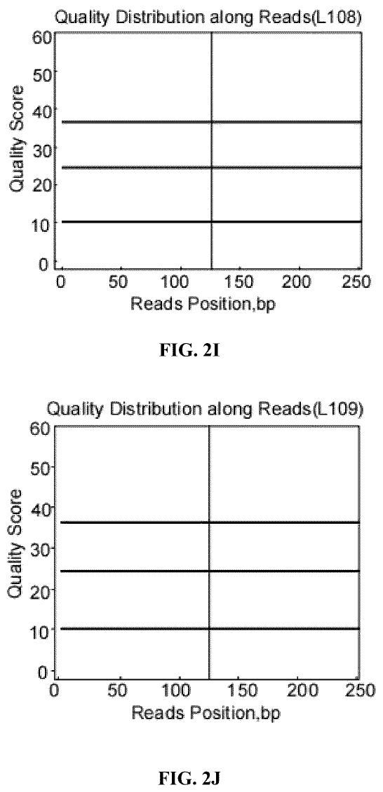

If the probability of base calling error is 0.001, the Q score of the base should be 30. The distribution of Q scores of the samples in this Example is shown in FIGS. 2A-2J, where the first 126 bp shows the distribution of Q scores of the first end reads of the paired-end reads, and the later 126 bp shows the distribution of Q scores of the other end reads. Each bp represents each base of all the reads, and the darker the color of each Q score at the same position is, the higher proportion of this Q score is in the data. For example, the first bp represents the distribution of Q score of the first base of all reads of the project in sequencing. It should be noted that only 10 plots are shown in FIGS. 2A-2J of this disclosure, but there are a total of 110 plots of sample Q score distribution, all of which can be viewed in BMK_slaf/Dataassess.

### (2) Base Distribution Check

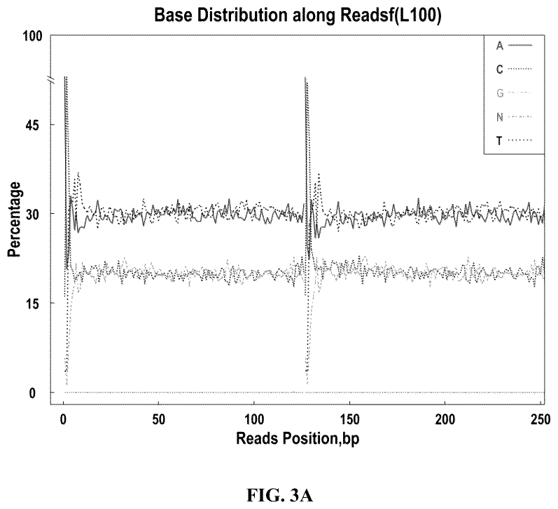

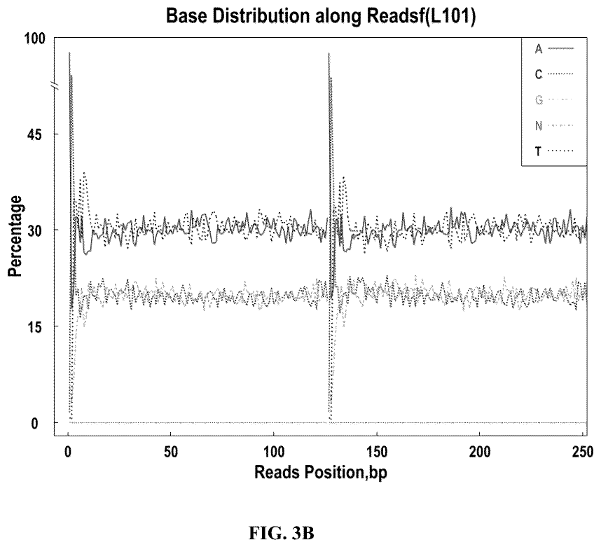

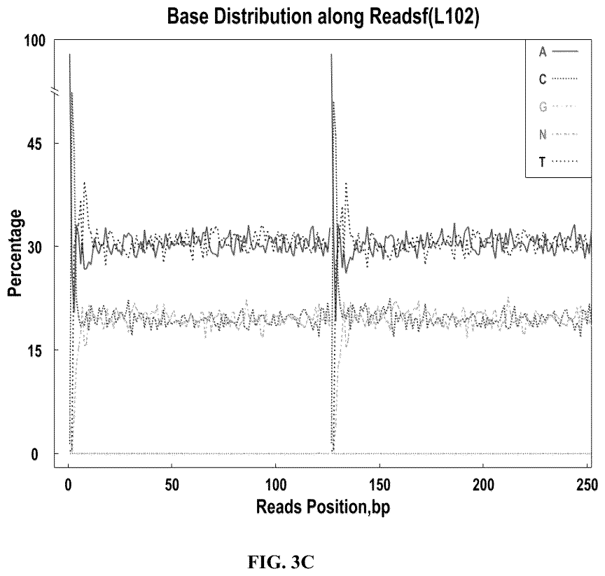

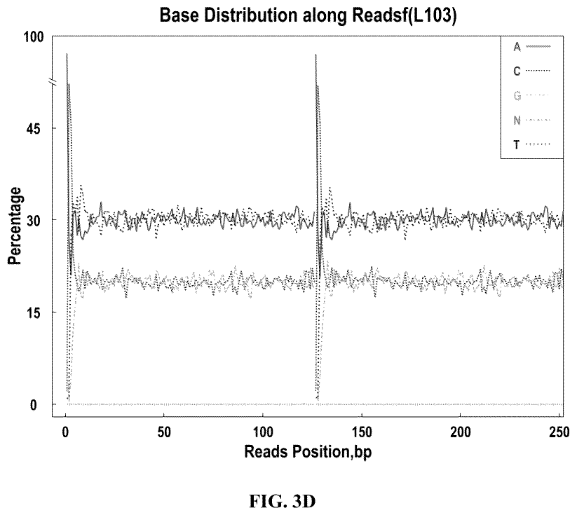

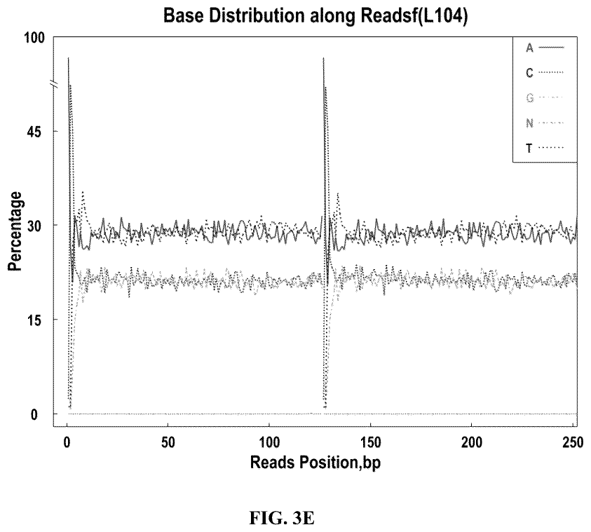

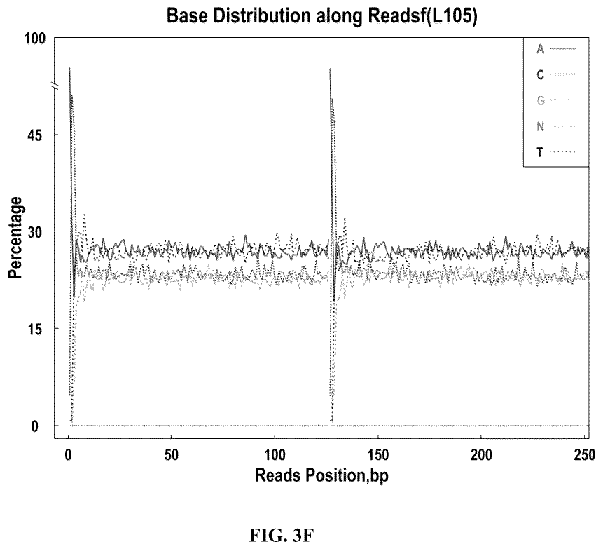

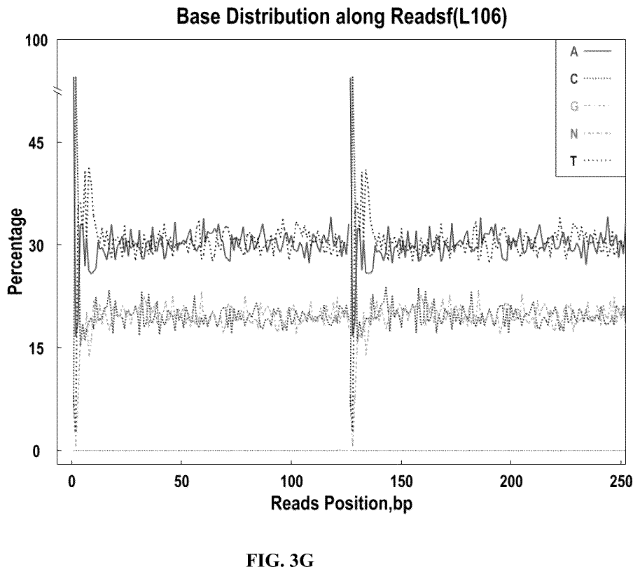

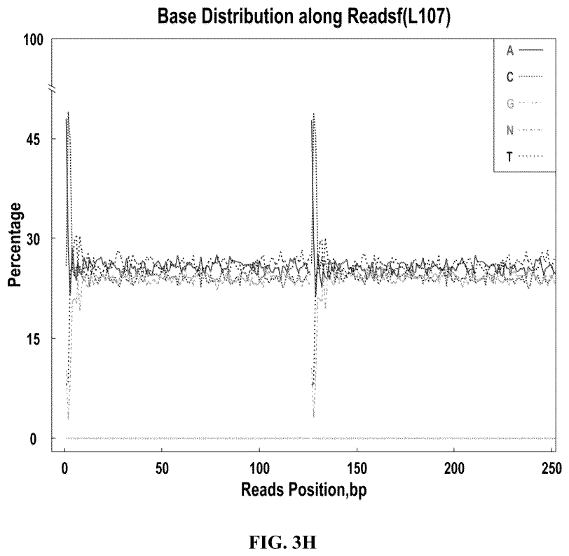

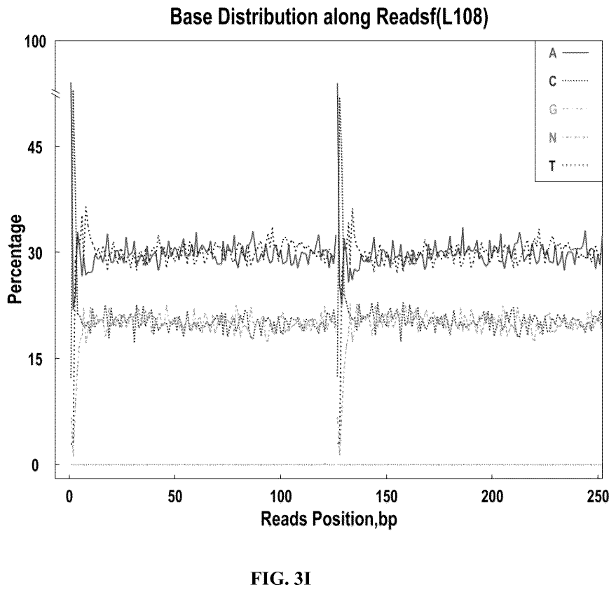

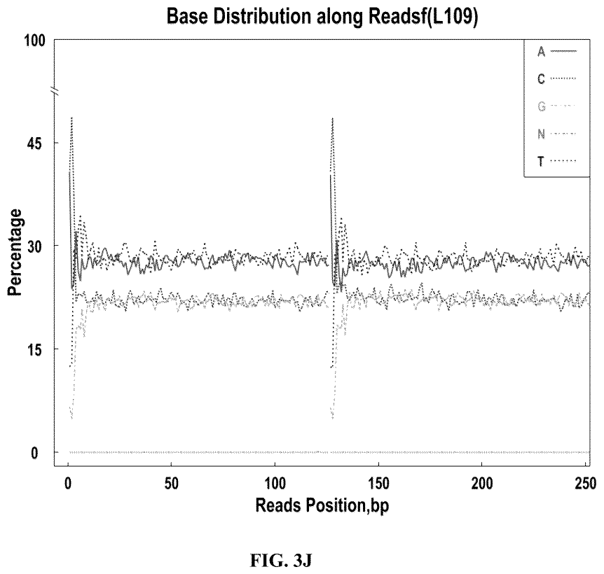

SLAF-seq reads are digested fragments of genomic DNA, and their base distribution is affected by the restriction site and PCR amplification. The first 2 bases of the reads will show base bias consistent with the restriction site, and the subsequent base distribution will show different degrees of fluctuation. The distribution of bases in this Example is shown in FIGS. 3A-3J, where different colors represent different base types: green represents base A, blue represents base T, red represents base C, orange represents base G, and gray represents base N that cannot be identified in sequencing. The first 126 bp shows the base distribution of the first end reads of the paired-end reads, and the later 126 bp shows the base distribution of the other end reads. Each bp represents each base of sequencing, e.g. the first bp means the distribution of A, T, G, C and N of all reads in the first base of the project. It should be noted that only 10 plots are shown in FIGS. 3A-3J, but there are a total of 110 plots of base distribution of sequenced samples in this disclosure, all of which can be viewed in BMK_slaf/Dataassess.

### 6. Information Analysis Process

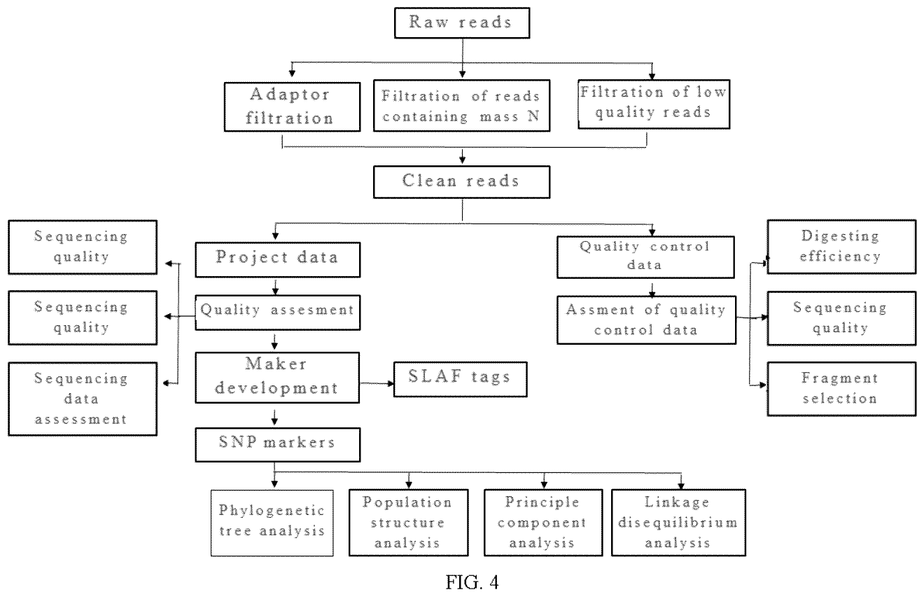

Bioinformatics analysis was performed on the resulting data after the evaluation of sequencing quality and data volume. Based on the bioinformatics analysis, genome-wide SNP markers were developed in the population, and population polymorphism analysis was performed using representative high-quality SNPs within the population. The specific bioinformatics analysis process is shown in FIG. 4.

### (1) SLAF Tag Development

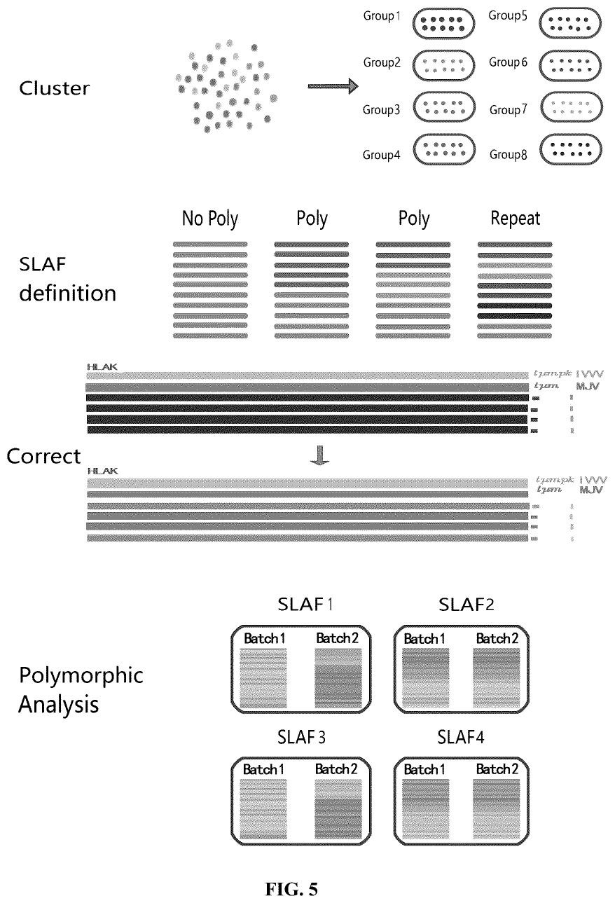

The reads generated by sequencing were derived from digested fragments of the same length produced by the same restriction endonuclease on different samples. The reads of each sample were clustered according to sequence similarity, and the reads clustered together were derived from one SLAF fragment (SLAF tag). The sequence similarity of the same SLAF tag between different samples was much higher than that between different SLAF tags; a SLAF tag was defined as a polymorphic SLAF tag when there was a sequence difference (i.e., there is polymorphism) between samples. The flow chart of polymorphic SLAF tag development is shown in FIG. 5.

According to the above method, the SLAF tags of 110 samples of Goji berry were finally obtained: each sample developed an average of 203,066 SLAF tags, and the average sequencing depth of the sample SLAF tags was 15.85×, with a guaranteed Q30 of 90%. A total of 425.00 Mb reads were obtained. 2,927,789 SLAF tags were obtained by bioinformatics analysis, of which a total of 75,905 SLAF tags were polymorphic, yielding a total of 1,441,595 population SNPs.

A total of 2,927,789 SLAF tags were developed in this example, and the average sequencing depth of the tags was 15.85×, and statistics of the SLAF tag are shown in Table 1.

TABLE 1
Statistics of SL4F tags

| Sample ID | BMK ID | SLAF number | Total depth | Average depth |
|---|---|---|---|---|
| L1 | L1 | 222,645 | 2,604,404 | 11.6976 |
| L10 | L10 | 134,069 | 1,458,189 | 10.8764 |
| L100 | L100 | 332,001 | 6,488,143 | 19.5425 |
| L101 | L101 | 288,331 | 5,546,966 | 19.2382 |
| L102 | L102 | 168,158 | 2,898,026 | 17.2339 |
| L103 | L103 | 204,930 | 4,113,112 | 20.0708 |
| L104 | L104 | 240,022 | 4,065,579 | 16.9384 |
| L105 | L105 | 150,536 | 5,146,648 | 34.1888 |
| L106 | L106 | 198,813 | 2,489,182 | 12.5202 |
| L107 | L107 | 208,250 | 3,600,778 | 17.2907 |
| L108 | L108 | 300,017 | 4,327,361 | 14.4237 |
| L109 | L109 | 160,586 | 2,715,027 | 16.9070 |
| L11 | L11 | 143,419 | 1,993,574 | 13.9003 |
| L110 | L110 | 268,703 | 4,935,964 | 18.3696 |
| L12 | L12 | 191,642 | 4,327,691 | 22.5822 |
| L13 | L13 | 172,244 | 2,646,426 | 15.3644 |
| L14 | L14 | 152,865 | 1,779,641 | 11.6419 |
| L15 | L15 | 239,019 | 8,354,836 | 34.9547 |
| L16 | L16 | 234,308 | 7,327,189 | 31.2716 |
| L17 | L17 | 245,329 | 3,343,216 | 13.6275 |
| L18 | L18 | 181,798 | 2,567,634 | 14.1236 |
| L19 | L19 | 205,056 | 3,634,829 | 17.7260 |
| L2 | L2 | 177,406 | 2,191,585 | 12.3535 |
| L20 | L20 | 264,786 | 5,923,671 | 22.3715 |
| L21 | L21 | 219,882 | 4,628,183 | 21.0485 |
| L22 | L22 | 258,061 | 4,737,859 | 18.3595 |
| L23 | L23 | 169,689 | 2,501,375 | 14.7409 |
| L24 | L24 | 162,783 | 1,942,130 | 11.9308 |
| L25 | L25 | 170,279 | 3,273,653 | 19.2252 |
| L26 | L26 | 196,948 | 3,302,550 | 16.7686 |
| L27 | L27 | 174,528 | 3,019,867 | 17.3031 |
| L28 | L28 | 151,490 | 2,472,594 | 16.3218 |
| L29 | L29 | 130,215 | 2,361,595 | 18.1361 |
| L3 | L3 | 148,710 | 1,433,569 | 9.6400 |
| L30 | L30 | 165,361 | 1,948,709 | 11.7846 |
| L31 | L31 | 128,031 | 1,220,751 | 9.5348 |
| L32 | L32 | 181,136 | 2,876,190 | 15.8786 |
| L33 | L33 | 141,721 | 1,532,113 | 10.8108 |
| L34 | L34 | 226,869 | 4,089,481 | 18.0257 |
| L35 | L35 | 204,119 | 3,230,319 | 15.8257 |
| L36 | L36 | 210,891 | 3,660,798 | 17.3587 |
| L37 | L37 | 152,842 | 2,396,200 | 15.6776 |
| L38 | L38 | 159,457 | 1,406,881 | 8.8229 |
| L39 | L39 | 157,821 | 1,933,297 | 12.2499 |
| L4 | L4 | 149,233 | 1,228,837 | 8.2344 |
| L40 | L40 | 55,871 | 1,534,974 | 27.4735 |
| L41 | L41 | 161,151 | 2,055,706 | 12.7564 |
| L42 | L42 | 67,321 | 3,256,249 | 48.3690 |
| L43 | L43 | 181,243 | 2,707,756 | 14.9399 |
| L44 | L44 | 171,582 | 1,673,401 | 9.7528 |
| L45 | L45 | 108,848 | 1,322,596 | 12.1509 |
| L46 | L46 | 162,550 | 2,058,664 | 12.6648 |
| L47 | L47 | 190,160 | 3,055,946 | 16.0704 |
| L48 | L48 | 171,932 | 2,454,571 | 14.2764 |
| L49 | L49 | 165,850 | 2,189,160 | 13.1996 |
| L5 | L5 | 189,153 | 1,730,375 | 9.1480 |
| L50 | L50 | 176,854 | 2,653,515 | 15.0040 |
| L51 | L51 | 208,781 | 4,273,380 | 20.4682 |
| L52 | L52 | 191,255 | 2,415,703 | 12.6308 |
| L53 | L53 | 194,912 | 3,119,295 | 16.0036 |
| L54 | L54 | 254,197 | 3,944,404 | 15.5171 |
| L55 | L55 | 209,955 | 3,898,769 | 18.5695 |
| L56 | L56 | 232,121 | 4,207,088 | 18.1245 |
| L57 | L57 | 222,729 | 3,738,978 | 16.7871 |
| L58 | L58 | 218,248 | 3,634,477 | 16.6530 |
| L59 | L59 | 188,240 | 2,925,407 | 15.5408 |
| L6 | L6 | 161,836 | 2,056,460 | 12.7071 |
| L60 | L60 | 391,062 | 19,889,618 | 50.8605 |
| L61 | L61 | 186,316 | 2,443,999 | 13.1175 |
| L62 | L62 | 210,525 | 3,148,095 | 14.9535 |
| L63 | L63 | 215,755 | 4,065,594 | 18.8436 |
| L64 | L64 | 298,829 | 5,282,689 | 17.6780 |
| L65 | L65 | 247,689 | 4,869,509 | 19.6598 |
| L66 | L66 | 197,982 | 2,625,722 | 13.2624 |
| L67 | L67 | 159,475 | 1,594,512 | 9.9985 |
| L68 | L68 | 236,627 | 4,884,553 | 20.6424 |
| L69 | L69 | 234,363 | 4,340,508 | 18.5204 |
| L7 | L7 | 175,160 | 1,947,463 | 11.1182 |
| L70 | L70 | 164,907 | 1,932,657 | 11.7197 |
| L71 | L71 | 218,427 | 2,107,730 | 9.6496 |
| L72 | L72 | 247,921 | 3,175,771 | 12.8096 |
| L73 | L73 | 208,201 | 2,298,330 | 11.0390 |
| L74 | L74 | 203,239 | 3,094,408 | 15.2255 |
| L75 | L75 | 245,122 | 5,606,830 | 22.8736 |
| L76 | L76 | 217,207 | 3,539,212 | 16.2942 |
| L77 | L77 | 223,291 | 3,760,971 | 16.8434 |
| L78 | L78 | 238,216 | 2,500,267 | 10.4958 |
| L79 | L79 | 227,331 | 4,684,219 | 20.6053 |
| L8 | L8 | 151,507 | 1,607,203 | 10.6081 |
| L80 | L80 | 331,721 | 4,228,797 | 12.7481 |
| L81 | L81 | 219,104 | 3,056,727 | 13.9510 |
| L82 | L82 | 225,543 | 4,143,893 | 18.3730 |
| L83 | L83 | 198,253 | 2,226,180 | 11.2290 |
| L84 | L84 | 268,736 | 5,247,056 | 19.5249 |
| L85 | L85 | 251,909 | 2,759,840 | 10.9557 |
| L86 | L86 | 210,268 | 3,750,893 | 17.8386 |
| L87 | L87 | 261,707 | 2,065,067 | 7.8908 |
| L88 | L88 | 210,160 | 1,544,267 | 7.3481 |
| L89 | L89 | 209,338 | 2,981,441 | 14.2422 |
| L9 | L9 | 156,479 | 1,571,738 | 10.0444 |
| L90 | L90 | 230,556 | 2,187,775 | 9.4891 |
| L91 | L91 | 233,514 | 2,851,178 | 12.2099 |
| L92 | L92 | 166,714 | 1,537,885 | 9.2247 |
| L93 | L93 | 247,932 | 2,223,133 | 8.9667 |
| L94 | L94 | 171,098 | 1,881,620 | 10.9973 |
| L95 | L95 | 233,837 | 2,615,151 | 11.1836 |
| L96 | L96 | 247,318 | 2,530,383 | 10.2313 |
| L97 | L97 | 208,878 | 1,983,958 | 9.4982 |
| L98 | L98 | 308,833 | 5,781,304 | 18.7198 |
| L99 | L99 | 216,475 | 3,570,567 | 16.4941 |

Note:
Sample ID: sample identifier; SL4F number: the number of SL4F tags contained in the corresponding samples; Total depth: the total depth of sequencing in SL4F tags of the corresponding samples, i.e. the total number of reads; Average depth: the average number of reads of the corresponding samples on each SL4F.

### (2) Acquisition and Statistics of SNP Markers

The sequence type with the highest depth in each SLAF tag was used as the reference sequence, and the reads were compared to the reference genome using bwa. SNPs were developed using both GATK and samtools, and the intersection of SNP markers obtained by the two methods was used as the final reliable SNP marker dataset. A total of 1,441,595 population SNPs were obtained. See Table 2 below:

TABLE 2
Statistics of SNPs in samples

| Sample ID | Total SNP | SNP num | Hetloci ratio(%) | Integrity ratio(%) |
|---|---|---|---|---|
| L1 | 1,441,595 | 594,642 | 5.39 | 41.24 |
| L10 | 1,441,595 | 344,203 | 5.43 | 23.87 |
| L100 | 1,441,595 | 498,394 | 7.74 | 34.57 |
| L101 | 1,441,595 | 474,649 | 8.43 | 32.92 |
| L102 | 1,441,595 | 390,285 | 6.05 | 27.07 |
| L103 | 1,441,595 | 425,519 | 4 | 29.51 |
| L104 | 1,441,595 | 402,445 | 6.44 | 27.91 |
| L105 | 1,441,595 | 179,427 | 2.5 | 12.44 |
| L106 | 1,441,595 | 353,999 | 6.39 | 24.55 |
| L107 | 1,441,595 | 12,202 | 0.58 | 0.84 |
| L108 | 1,441,595 | 473,270 | 6.73 | 32.82 |
| L109 | 1,441,595 | 14,928 | 0.44 | 1.03 |
| L11 | 1,441,595 | 355,211 | 6.82 | 24.64 |
| L110 | 1,441,595 | 460,964 | 7.53 | 31.97 |
| L12 | 1,441,595 | 517,762 | 6.73 | 35.91 |
| L13 | 1,441,595 | 470,575 | 6.01 | 32.64 |
| L14 | 1,441,595 | 414,896 | 4.83 | 28.78 |
| L15 | 1,441,595 | 610,245 | 9.13 | 42.33 |
| L16 | 1,441,595 | 600,118 | 8.43 | 41.62 |
| L17 | 1,441,595 | 627,051 | 5.23 | 43.49 |
| L18 | 1,441,595 | 462,369 | 6.85 | 32.07 |
| L19 | 1,441,595 | 520,745 | 7.4 | 36.12 |
| L2 | 1,441,595 | 455,226 | 7 | 31.57 |
| L20 | 1,441,595 | 665,403 | 8.62 | 46.15 |
| L21 | 1,441,595 | 138,159 | 3.12 | 9.58 |
| L22 | 1,441,595 | 664,671 | 7.84 | 46.1 |
| L23 | 1,441,595 | 461,510 | 6.66 | 32.01 |
| L24 | 1,441,595 | 436,417 | 4.99 | 30.27 |
| L25 | 1,441,595 | 440,509 | 5.3 | 30.55 |
| L26 | 1,441,595 | 522,680 | 6.22 | 36.25 |
| L27 | 1,441,595 | 454,993 | 6.04 | 31.56 |
| L28 | 1,441,595 | 401,499 | 2.71 | 27.85 |
| L29 | 1,441,595 | 75,224 | 1.59 | 5.21 |
| L3 | 1,441,595 | 392,544 | 4.78 | 27.22 |
| L30 | 1,441,595 | 444,669 | 4.85 | 30.84 |
| L31 | 1,441,595 | 310,447 | 6.07 | 21.53 |
| L32 | 1,441,595 | 459,774 | 5.53 | 31.89 |
| L33 | 1,441,595 | 370,255 | 3.86 | 25.68 |
| L34 | 1,441,595 | 532,368 | 9.09 | 36.92 |
| L35 | 1,441,595 | 460,973 | 6.29 | 31.97 |
| L36 | 1,441,595 | 529,619 | 7.6 | 36.73 |
| L37 | 1,441,595 | 385,755 | 6.19 | 26.75 |
| L38 | 1,441,595 | 414,953 | 4.63 | 28.78 |
| L39 | 1,441,595 | 412,607 | 5.56 | 28.62 |
| L4 | 1,441,595 | 371,789 | 4.98 | 25.79 |
| L40 | 1,441,595 | 8,264 | 0.41 | 0.57 |
| L41 | 1,441,595 | 307,131 | 3.85 | 21.3 |
| L42 | 1,441,595 | 15,843 | 0.47 | 1.09 |
| L43 | 1,441,595 | 482,398 | 6.92 | 33.46 |
| L44 | 1,441,595 | 467,959 | 4.66 | 32.46 |
| L45 | 1,441,595 | 219,573 | 2.69 | 15.23 |
| L46 | 1,441,595 | 214,845 | 4.12 | 14.9 |
| L47 | 1,441,595 | 497,532 | 6.82 | 34.51 |
| L48 | 1,441,595 | 453,637 | 6.31 | 31.46 |
| L49 | 1,441,595 | 438,095 | 7.94 | 30.38 |
| L5 | 1,441,595 | 513,500 | 5.15 | 35.62 |
| L50 | 1,441,595 | 345,281 | 5.07 | 23.95 |
| L51 | 1,441,595 | 552,612 | 7.94 | 38.33 |
| L52 | 1,441,595 | 423,254 | 4.82 | 29.36 |
| L53 | 1,441,595 | 516,606 | 5.81 | 35.83 |
| L54 | 1,441,595 | 595,588 | 6.58 | 41.31 |
| L55 | 1,441,595 | 537,212 | 5.72 | 37.26 |
| L56 | 1,441,595 | 581,953 | 7.93 | 40.36 |
| L57 | 1,441,595 | 578,381 | 7.78 | 40.12 |
| L58 | 1,441,595 | 542,810 | 5.94 | 37.65 |
| L59 | 1,441,595 | 513,336 | 5.34 | 35.6 |
| L6 | 1,441,595 | 403,981 | 5.94 | 28.02 |
| L60 | 1,441,595 | 811,577 | 10.56 | 56.29 |
| L61 | 1,441,595 | 485,100 | 6.01 | 33.65 |
| L62 | 1,441,595 | 537,897 | 6.6 | 37.31 |
| L63 | 1,441,595 | 567,532 | 6.68 | 39.36 |
| L64 | 1,441,595 | 473,436 | 5.84 | 32.84 |
| L65 | 1,441,595 | 549,028 | 7.1 | 38.08 |
| L66 | 1,441,595 | 492,005 | 6.56 | 34.12 |
| L67 | 1,441,595 | 419,506 | 4.39 | 29.1 |
| L68 | 1,441,595 | 598,391 | 6.82 | 41.5 |
| L69 | 1,441,595 | 587,803 | 6.71 | 40.77 |
| L7 | 1,441,595 | 427,861 | 8.61 | 29.67 |
| L70 | 1,441,595 | 440,970 | 5.05 | 30.58 |
| L71 | 1,441,595 | 511,630 | 5.37 | 35.49 |
| L72 | 1,441,595 | 573,677 | 6.63 | 39.79 |
| L73 | 1,441,595 | 530,181 | 6.55 | 36.77 |
| L74 | 1,441,595 | 526,342 | 6.37 | 36.51 |
| L75 | 1,441,595 | 613,877 | 7.67 | 42.58 |
| L76 | 1,441,595 | 558,473 | 7.69 | 38.73 |
| L77 | 1,441,595 | 555,118 | 7.09 | 38.5 |
| L78 | 1,441,595 | 354,871 | 4.95 | 24.61 |
| L79 | 1,441,595 | 611,522 | 7.12 | 42.41 |
| L8 | 1,441,595 | 392,272 | 5.32 | 27.21 |
| L80 | 1,441,595 | 484,463 | 5.89 | 33.6 |
| L81 | 1,441,595 | 144,601 | 3.19 | 10.03 |
| L82 | 1,441,595 | 560,844 | 6.23 | 38.9 |
| L83 | 1,441,595 | 474,384 | 6.31 | 32.9 |
| L84 | 1,441,595 | 651,360 | 6.58 | 45.18 |
| L85 | 1,441,595 | 393,779 | 4.84 | 27.31 |
| L86 | 1,441,595 | 539,169 | 6.38 | 37.4 |
| L87 | 1,441,595 | 555,949 | 6.04 | 38.56 |
| L88 | 1,441,595 | 505,102 | 6.28 | 35.03 |
| L89 | 1,441,595 | 520,633 | 6.95 | 36.11 |
| L9 | 1,441,595 | 396,930 | 4.99 | 27.53 |
| L90 | 1,441,595 | 580,281 | 7.2 | 40.25 |
| L91 | 1,441,595 | 581,935 | 6 | 40.36 |
| L92 | 1,441,595 | 420,529 | 4.42 | 29.17 |
| L93 | 1,441,595 | 571,240 | 7.32 | 39.62 |
| L94 | 1,441,595 | 433,164 | 4.9 | 30.04 |
| L95 | 1,441,595 | 541,955 | 6.61 | 37.59 |
| L96 | 1,441,595 | 591,464 | 7.12 | 41.02 |
| L97 | 1,441,595 | 478,360 | 5.73 | 33.18 |
| L98 | 1,441,595 | 408,862 | 6.08 | 28.36 |
| L99 | 1,441,595 | 419,015 | 7.86 | 29.06 |

Note:
Sample ID: sample identifier; Total SNP: total number of SNPs detected; SNP num: number of SNPs detected in the corresponding sample; Integrity ratio: integrity of SNPs detected in the sample; Hetloci ratio: ratio of heterozygous SNPs in the sample.

### Example 2

### Genetic Analysis Based on the SNPs Obtained in Example 1

### (1) Phylogenetic Tree Analysis

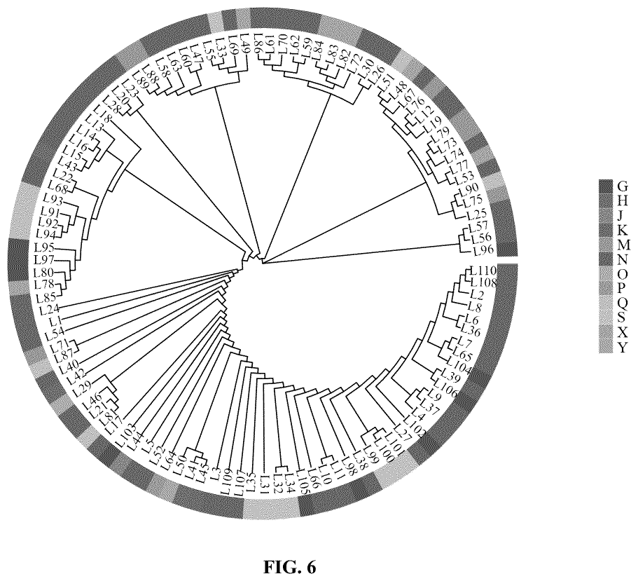

MEGA X software was used to construct the phylogenetic tree of the samples, and the neighbor-joining method and the Kimura 2-parameter model were adopted, with 1000 bootstrap iterations. The results are shown in FIGS. 6A-6C.

It was shown that the 110 Goji berry germplasms were divided into five major genetic taxa, and taxon I could be further divided into three subtaxa. Among them, subtaxon 1 mainly included samples from the first and second primitive ancestral groups and a few from the third ancestral group, mainly including bitter, semi-bitter Gooji berries, etc.; subtaxa 2 and 3 were the transitional types in the evolution of bitter to sweet Goji berries. Taxa II, III, VI, and V belonged to the third primitive ancestral group, which are different taxa evolving into sweet Goji berry.

### (2) Population PCA

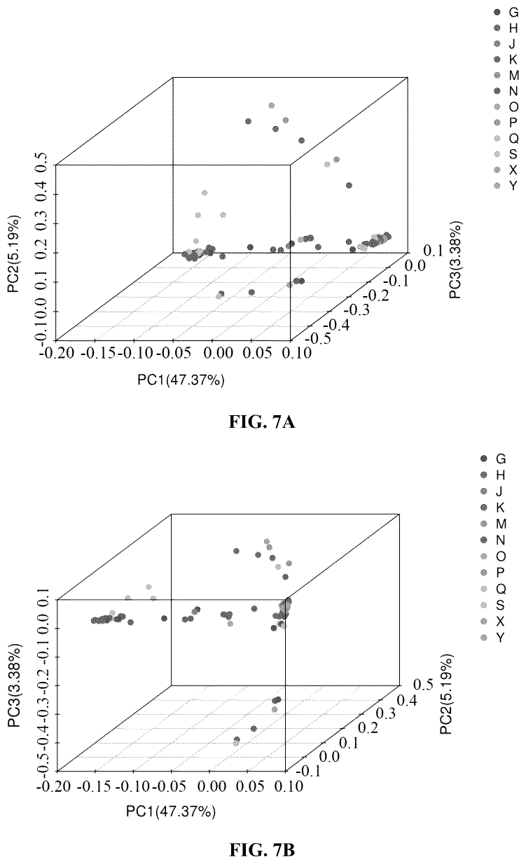

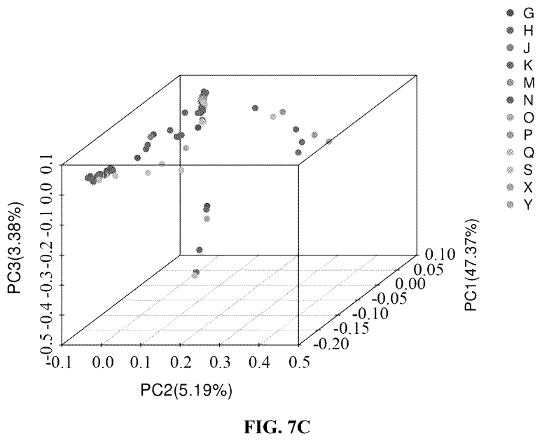

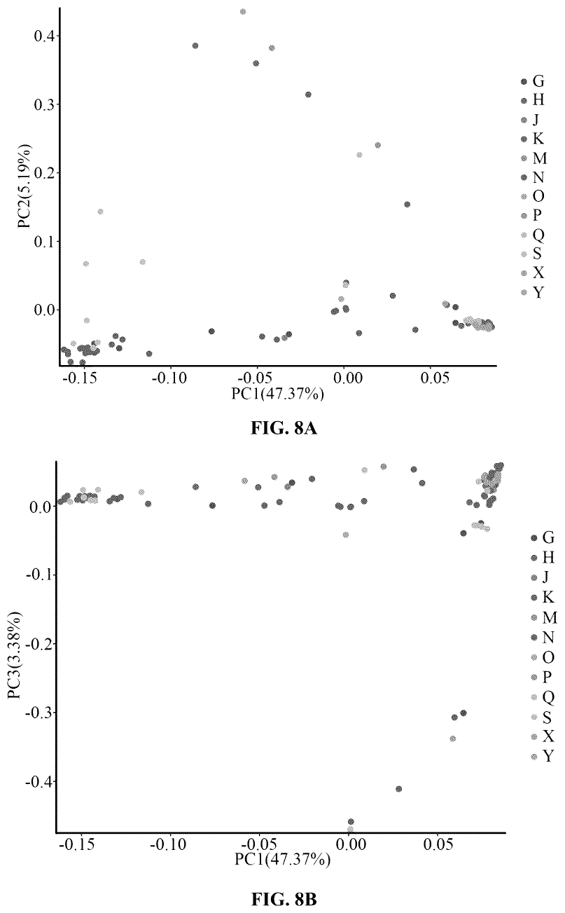

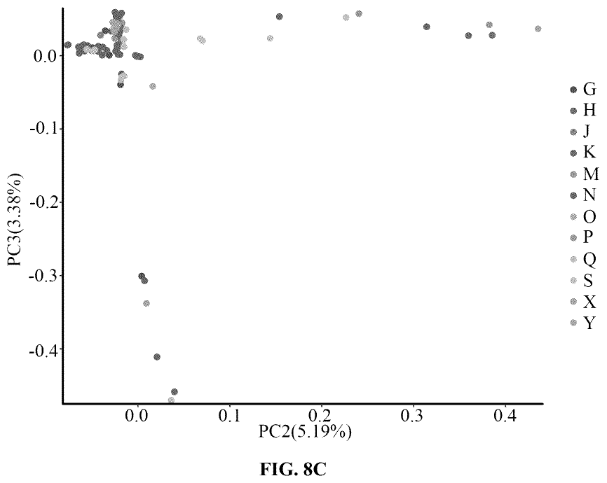

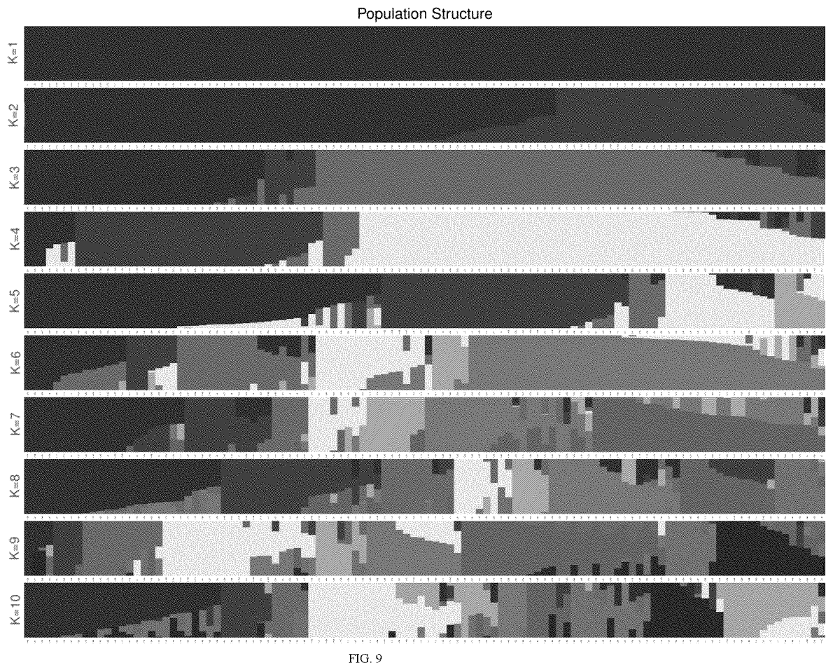

Based on SNP data, PCA was performed with EIGENSOFT software to obtain the clustering of the samples. The clustering by PCA is shown in FIGS. 7A-7C and FIG. 8, where the samples are clustered on two dimensions by PCA, PC1 represents the first principal component, PC2 represents the second principal component; PC3 represents the third principal component. A dot represents a sample, and a color represents a group.

### (3) Population Structure Analysis

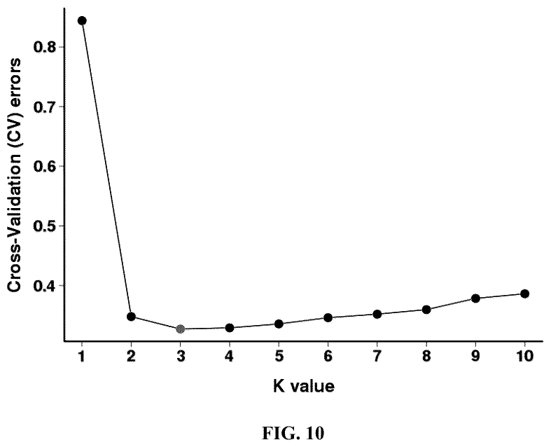

Based on the SNPs obtained in Example 1, the population structure of the Goji berry material was analyzed using admixture software. The number of subgroups (K values) was pre-set to 1-10 for clustering (FIG. 9), and the clustering results were cross-validated to determine the optimal number of subgroups according to the valley of the cross-validation error rate. The clustering for K values of 1-10 and the cross-validation error rate corresponding to each K value are shown in FIG. 10.

The relationship of the 110 samples with the populations are shown in Table 3:

TABLE 3
Correspondence between the 110 samples of
Goji berry germpL4sms and sub-popuL4tions

| Sample ID | BMK ID | Q1 | Q2 | Q3 | Group |
|---|---|---|---|---|---|
| L1 | L1 | 0.293197 | 0.000010 | 0.706793 | Q3 |
| L10 | L10 | 0.999980 | 0.000010 | 0.000010 | Q1 |
| L100 | L100 | 0.999980 | 0.000010 | 0.000010 | Q1 |
| L101 | L101 | 0.999980 | 0.000010 | 0.000010 | Q1 |
| L102 | L102 | 0.999980 | 0.000010 | 0.000010 | Q1 |
| L103 | L103 | 0.484167 | 0.000010 | 0.515823 | Q3 |
| L104 | L104 | 0.999980 | 0.000010 | 0.000010 | Q1 |
| L105 | L105 | 0.999980 | 0.000010 | 0.000010 | Q1 |
| L106 | L106 | 0.999980 | 0.000010 | 0.000010 | Q1 |
| L107 | L107 | 0.999980 | 0.000010 | 0.000010 | Q1 |
| L108 | L108 | 0.999980 | 0.000010 | 0.000010 | Q1 |
| L109 | L109 | 0.999980 | 0.000010 | 0.000010 | Q1 |
| L11 | L11 | 0.999980 | 0.000010 | 0.000010 | Q1 |
| L110 | L110 | 0.999980 | 0.000010 | 0.000010 | Q1 |
| L12 | L12 | 0.000010 | 0.000010 | 0.999980 | Q3 |
| L13 | L13 | 0.000010 | 0.000010 | 0.999980 | Q3 |
| L14 | L14 | 0.000010 | 0.000010 | 0.999980 | Q3 |
| L15 | L15 | 0.000010 | 0.000010 | 0.999980 | Q3 |
| L16 | L16 | 0.000010 | 0.000010 | 0.999980 | Q3 |
| L17 | L17 | 0.493246 | 0.000010 | 0.506744 | Q3 |
| L18 | L18 | 0.000010 | 0.000010 | 0.999980 | Q3 |
| L19 | L19 | 0.000010 | 0.000010 | 0.999980 | Q3 |
| L2 | L2 | 0.999980 | 0.000010 | 0.000010 | Q1 |
| L20 | L20 | 0.000010 | 0.000010 | 0.999980 | Q3 |
| L21 | L21 | 0.000010 | 0.699475 | 0.300515 | Q2 |
| L22 | L22 | 0.000010 | 0.000010 | 0.999980 | Q3 |
| L23 | L23 | 0.000010 | 0.000010 | 0.999980 | Q3 |
| L24 | L24 | 0.149775 | 0.000010 | 0.850215 | Q3 |
| L25 | L25 | 0.000010 | 0.000010 | 0.999980 | Q3 |
| L26 | L26 | 0.000010 | 0.000010 | 0.999980 | Q3 |
| L27 | L27 | 0.999980 | 0.000010 | 0.000010 | Q1 |
| L28 | L28 | 0.000010 | 0.000010 | 0.999980 | Q3 |
| L29 | L29 | 0.087227 | 0.444194 | 0.468579 | Q3 |
| L3 | L3 | 0.886984 | 0.000010 | 0.113006 | Q1 |
| L30 | L30 | 0.000010 | 0.000010 | 0.999980 | Q3 |
| L31 | L31 | 0.848110 | 0.151880 | 0.000010 | Q1 |
| L32 | L32 | 0.956393 | 0.043597 | 0.000010 | Q1 |
| L33 | L33 | 0.000010 | 0.000010 | 0.999980 | Q3 |
| L34 | L34 | 0.845661 | 0.154329 | 0.000010 | Q1 |
| L35 | L35 | 0.785515 | 0.214475 | 0.000010 | Q1 |
| L36 | L36 | 0.999980 | 0.000010 | 0.000010 | Q1 |
| L37 | L37 | 0.999980 | 0.000010 | 0.000010 | Q1 |
| L38 | L38 | 0.999980 | 0.000010 | 0.000010 | Q1 |
| L39 | L39 | 0.999980 | 0.000010 | 0.000010 | Q1 |
| L4 | L4 | 0.999980 | 0.000010 | 0.000010 | Q1 |
| L40 | L40 | 0.000010 | 0.321992 | 0.677998 | Q3 |
| L41 | L41 | 0.201680 | 0.798310 | 0.000010 | Q2 |
| L42 | L42 | 0.190663 | 0.086773 | 0.722564 | Q3 |
| L43 | L43 | 0.034896 | 0.000010 | 0.965094 | Q3 |
| L44 | L44 | 0.492014 | 0.000010 | 0.507976 | Q3 |
| L45 | L45 | 0.000010 | 0.999980 | 0.000010 | Q2 |
| L46 | L46 | 0.000010 | 0.310812 | 0.689178 | Q3 |
| L47 | L47 | 0.000010 | 0.000010 | 0.999980 | Q3 |
| L48 | L48 | 0.000010 | 0.000010 | 0.999980 | Q3 |
| L49 | L49 | 0.000010 | 0.000010 | 0.999980 | Q3 |
| L5 | L5 | 0.535738 | 0.000010 | 0.464252 | Q1 |
| L50 | L50 | 0.000010 | 0.999980 | 0.000010 | Q2 |
| L51 | L51 | 0.000010 | 0.000010 | 0.999980 | Q3 |
| L52 | L52 | 0.000010 | 0.692093 | 0.307897 | Q2 |
| L53 | L53 | 0.000010 | 0.000010 | 0.999980 | Q3 |
| L54 | L54 | 0.000010 | 0.307243 | 0.692747 | Q3 |
| L55 | L55 | 0.000010 | 0.000010 | 0.999980 | Q3 |
| L56 | L56 | 0.000010 | 0.000010 | 0.999980 | Q3 |
| L57 | L57 | 0.000010 | 0.000010 | 0.999980 | Q3 |
| L58 | L58 | 0.000010 | 0.000010 | 0.999980 | Q3 |
| L59 | L59 | 0.000010 | 0.000010 | 0.999980 | Q3 |
| L6 | L6 | 0.999980 | 0.000010 | 0.000010 | Q1 |
| L60 | L60 | 0.000010 | 0.000010 | 0.999980 | Q3 |
| L61 | L61 | 0.000010 | 0.000010 | 0.999980 | Q3 |
| L62 | L62 | 0.000010 | 0.000010 | 0.999980 | Q3 |
| L63 | L63 | 0.000010 | 0.000010 | 0.999980 | Q3 |
| L64 | L64 | 0.000010 | 0.813832 | 0.186158 | Q2 |
| L65 | L65 | 0.999980 | 0.000010 | 0.000010 | Q1 |
| L66 | L66 | 0.959551 | 0.000010 | 0.040439 | Q1 |
| L67 | L67 | 0.000010 | 0.000010 | 0.999980 | Q3 |
| L68 | L68 | 0.000010 | 0.000010 | 0.999980 | Q3 |
| L69 | L69 | 0.000010 | 0.000010 | 0.999980 | Q3 |
| L7 | L7 | 0.999980 | 0.000010 | 0.000010 | Q1 |
| L70 | L70 | 0.000010 | 0.000010 | 0.999980 | Q3 |
| L71 | L71 | 0.000010 | 0.428310 | 0.571680 | Q3 |
| L72 | L72 | 0.000010 | 0.000010 | 0.999980 | Q3 |
| L73 | L73 | 0.000010 | 0.000010 | 0.999980 | Q3 |
| L74 | L74 | 0.000010 | 0.000010 | 0.999980 | Q3 |
| L75 | L75 | 0.000010 | 0.000010 | 0.999980 | Q3 |
| L76 | L76 | 0.000010 | 0.000010 | 0.999980 | Q3 |
| L77 | L77 | 0.000010 | 0.000010 | 0.999980 | Q3 |
| L78 | L78 | 0.000010 | 0.155133 | 0.844857 | Q3 |
| L79 | L79 | 0.000010 | 0.000010 | 0.999980 | Q3 |
| L8 | L8 | 0.999980 | 0.000010 | 0.000010 | Q1 |
| L80 | L80 | 0.000010 | 0.137241 | 0.862749 | Q3 |
| L81 | L81 | 0.000010 | 0.675620 | 0.324370 | Q2 |
| L82 | L82 | 0.000010 | 0.000010 | 0.999980 | Q3 |
| L83 | L83 | 0.000010 | 0.000010 | 0.999980 | Q3 |
| L84 | L84 | 0.000010 | 0.000010 | 0.999980 | Q3 |
| L85 | L85 | 0.000010 | 0.153709 | 0.846281 | Q3 |
| L86 | L86 | 0.000010 | 0.000010 | 0.999980 | Q3 |
| L87 | L87 | 0.000010 | 0.476368 | 0.523622 | Q3 |
| L88 | L88 | 0.000010 | 0.010455 | 0.989535 | Q3 |
| L89 | L89 | 0.000010 | 0.000010 | 0.999980 | Q3 |
| L9 | L9 | 0.999980 | 0.000010 | 0.000010 | Q1 |
| L90 | L90 | 0.000010 | 0.011924 | 0.988066 | Q3 |
| L91 | L91 | 0.000010 | 0.000010 | 0.999980 | Q3 |
| L92 | L92 | 0.000010 | 0.000010 | 0.999980 | Q3 |
| L93 | L93 | 0.000010 | 0.000010 | 0.999980 | Q3 |
| L94 | L94 | 0.000010 | 0.000010 | 0.999980 | Q3 |
| L95 | L95 | 0.000010 | 0.010163 | 0.989827 | Q3 |
| L96 | L96 | 0.000010 | 0.000010 | 0.999980 | Q3 |
| L97 | L97 | 0.038391 | 0.009807 | 0.951803 | Q3 |
| L98 | L98 | 0.999980 | 0.000010 | 0.000010 | Q1 |
| L99 | L99 | 0.999980 | 0.000010 | 0.000010 | Q1 |

Note:
Sample ID: sample identifier; BMK ID: the unified identifier for the project samples designated by Biomarker Co. Ltd.; Q1: the likelihood that the sample is from the first primitive ancestry; Q2: the likelihood that the sample is from the second primitive ancestry; Q3: the likelihood that the sample is from the third primitive ancestry; Group: sample group.

The analysis results are shown Table 4.

TABLE 4
Summary of primitive ancestrys of clusters in genetic evolutionary tree of Goji berry germplasms

| | | | | Collection | Latitude | Longtitude | | Sweet or | Bitter | Probability of |
|---|---|---|---|---|---|---|---|---|---|---|
| Group | No. | Code | Name | date | (N) | (E) | Height | bitter taste | index | primitive ancestry |
| Taxon I | L110 | N | Ziku No. 3 from Limei Village, Zhenhu Township, | 2021 Sep. 19 | 35.8479 | 105.4761 | 1824 | Bitter | 10 | Q1 |
| (subtaxon I) | | | Xiji County | | | | | | | |
| | L108 | N | Ziku 1 fromLimei Village, Zhenhu Township, Xiji | 2021 Sep. 19 | 35.8477 | 105.4759 | 1818 | Bitter | 10 | Q1 |
| | | | County | | | | | | | |
| | L2 | N | Dongyehong No. 2 from Zhenhu Road, Xiji County | 2019 Sep. 22 | 35.8457 | 105.4641 | 1834 | Bitter | 10 | Q1 |
| | L8 | N | Dongyezi No. 2 from Zhenhu Road, Xiji County | 2019 Sep. 22 | 35.8457 | 105.4641 | 1835 | Bitter | 10 | Q1 |
| | L6 | N | Yehong from Shacongwa, Jiqiang Town, Xiji | 2014 Aug. 26 | 36.0100 | 105.6806 | 2004 | Medium | 7 | Q1 |
| | | | County | | | | | bitter | | |
| | L36 | N | Longzhang Highway, Tianbao Village, Yuanzhou | 2020 Jun. 7 | 35.8040 | 106.1080 | 2085 | Bitter | 10 | Q1 |
| | | | District, Guyuan City | | | | | | | |
| | L7 | N | Yehong No. 1 from Yuanhe Village, Xi’an Town, | 2014 Oct. 27 | 36.5921\grave{ } | 105.4530 | 1752 | Bitter | 10 | Q1 |
| | | | Haiyuan County | | | | | | | |
| | L65 | N | Yehong No. 2 from Yuanhe Village, Xi’an Town, | 2014 Aug. 26 | 36.5921 | 105.4530 | 1752 | Bitter | 10 | Q1 |
| | | | Haiyuan County | | | | | | | |
| | L104 | G | Baicaowa Village No. 2 from Wugou Township, | 2021 Sep. 4 | 35.9112 | 106.8848 | 1518 | Bitter | 10 | Q1 |
| | | | Zhenyuan County, Gansu Province | | | | | | | |
| | L39 | N | Yehongku No.1 from Haoshui Gas Station of | 2020 Jun. 7 | 35.6591 | 106.1051 | 2049 | Bitter | 10 | Q1 |
| | | | Longzhang Highway, Longde County | | | | | | | |
| | L106 | G | Baicaowa Village, Wugou Township, Zhenyuan | 2021 Sep. 4 | 35.9081 | 106.8771 | 1603 | Bitter | 10 | Q1 |
| | | | County, Gansu Province | | | | | | | |
| | L9 | N | Yehong, from Yanziwan Village, Honghe Township, | 2019 Sep. 23 | 106.6875 | 35.7516 | 1601 | Bitter | 10 | Q1 |
| | | | Pengyang County | | | | | | | |
| | L37 | N | Quanku Goji berry from Zhongzhuang Village, | 2018 Sep. 11 | 35.9337 | 106.7184 | 1600 | Bitter | 10 | Q1 |
| | | | Pengyang County | | | | | | | |
| | L4 | N | Zhangjiawan, Yashanliang, Mourong Village, | 2010 Sep. 22 | 35.8282 | 105.8574 | 1869 | Bitter | 10 | Q1 |
| | | | Jiangtaibao, Xiji County | | | | | | | |
| | L102 | G | Baicaowa Village, Wugou Township, Zhenyuan | 2021 Sep. 4 | 35.9081 | 106.8771 | 1603 | Spicy and | 10 | Q1 |
| | | | County, Gansu Province | | | | | bitter | | |
| | L27 | N | Yehong from Zhengjue Temple, Wangquangou, | 2019 Oct. 16 | 39.1447 | 106.5479 | 1253 | Bitter and | 8 | Q1 |
| | | | Huinong District, Shizuishan | | | | | salty | | |
| | L101 | S | Yehong NO. 3 from Qiaogetai Village, Taozhen | 2021 Aug. 31 | 37.7346 | 110.4011 | 1193 | Bitter | 10 | Q1 |
| | | | Town, Mizhi County, Shaanxi Province | | | | | | | |
| | L100 | S | Yehong No. 2 from Qiaogetai Village, Taozhen | 2021 Aug. 31 | 37.7346 | 110.4011 | 1193 | Bitter | 10 | Q1 |
| | | | Town, Mizhi County, Shaanxi Province | | | | | | | |
| | L99 | S | Yehong No. 1 from Qiaogetai Village, Taozhen | 2021 Aug. 31 | 37.7397 | 110.4081 | 1087 | Bitter | 10 | Q1 |
| | | | Town, Mizhi County, Shaanxi Province | | | | | | | |
| | L38 | N | Quanku Goji berry from Helan Mountain Yanhua | 2017 Aug. 17 | 38.7329 | 106.0145 | 1414 | Bitter | 10 | Q1 |
| | L98 | G | No. 100 County Road near Lijiamen Village, | 2020 Aug. 7 | 34.9561 | 105.1052 | 1689 | Bitter | 10 | Q1 |
| | | | Gangu County, Tianshui City, Gansu Province | | | | | | | |
| | L11 | N | Renshanhe, Pengyang County | 2019 Jul. 26 | 35.87551 | 106.4000 | 1722 | Bitter | 10 | Q1 |
| | L10 | N | Xiaocha Team, Zhongzhuang Village, Baiyang | 2019 Sep. 23 | 35.2162 | 106.7065 | 1671 | Bitter | 10 | Q1 |
| | | | Town, Pengyang County | | | | | | | |
| | L66 | N | Yehong No. 3 from Yuanhe Village, Xi’an Town, | 2014 Aug. 26 | 36.59213 | 105.4530 | 1752 | Bitter | 10 | Q1 |
| | | | Haiyuan County | | | | | | | |
| | L105 | G | Bai Grassland, Baicaowa Village, Wugou | 2021 Sep. 4 | 35.9109 | 106.8849 | 1514 | Medium | 5 | Q1 |
| | | | Township, Zhenyuan County, Gansu Province | | | | | bitter | | |
| | L34 | S | Yehong Goji berry from Zhujia Lane, Fengming | 2019 Nov. 7 | 34.4836 | 107.5992 | 730 | Bitter | 10 | Q1 |
| | | | Town, Qishan County, Shaanxi Province | | | | | | | |
| | L32 | S | Yehong Goji berry from east of Shaanxi Zhouyuan | 2019 Nov. 7 | 34.4822 | 107.8661 | 674.2 | Bitter | 10 | Q1 |
| | | | Museum | | | | | | | |
| | L31 | S | Ye Hong from Fujiazu, Jiaoliu Village, Qinghua | 2019 Nov. 7 | 34.4653 | 107.8133 | 701.2 | Bitter | 10 | Q1 |
| | | | Town, Qishan County, Shaanxi Province | | | | | | | |
| | L35 | S | Ye Hong from Xishan (Beishan), Kushan Village, | 2019 Nov. 7 | 34.5127 | 107.7400 | 891.3 | Bitter | 10 | Q1 |
| | | | Pucun Town, Qishan County, Shaanxi Province | | | | | | | |
| | L107 | N | Guanqiao Township, Haiyuan County is bitter and | 2021 Sep. 18 | 36.7498 | 105.7672 | 1526 | Bitter and | 10 | Q1 |
| | | | slightly spicy | | | | | mild spicy | | |
| | L109 | N | Ziku No. 2 from Limei Village, Zhenhu Township, | 2021 Sep. 19 | 35.8479 | 105.4762 | 1825 | Bitter | 10 | Q1 |
| | | | Xiji County | | | | | | | |
| | L3 | N | Yezi No. 1 from east of Xiji Zhenhu Road | 2019 Sep. 22 | 35.8457 | 105.4641 | 1835 | Bitter | 10 | Q1 |
| | L45 | K | Guangdong broadleaf | 2020 Jul. 8 | 38.6466 | 106.1531 | 1054 | Mild sweet | 0 | Q2 |
| | L41 | H | Yehong No. 1 from Xinxiang, Henan | 2020 Jul. 8 | 38.5147 | 106.2358 | 1056 | Medium | 6 | Q2 |
| | | | | | | | | bitter | | |
| | L50 | Y | Lycium yunnanense | 2020 Jul. 8 | 38.6466 | 106.1531 | 1054 | Sweet | 0 | Q2 |
| | L64 | P | Korean Goji berry | 2020 Jul. 8 | 38.5147 | 106.2358 | 1056 | Medium | 6 | Q2 |
| | | | | | | | | bitter | | |
| | L52 | N | Chinese Goji berry | 2020 Jul. 8 | 38.6466 | 106.1531 | 1054 | Sweet | 0 | Q2 |
| | L5 | N | Wild Chinese Goji berry from Tianping Township, | 2013 Sep. 16 | 35.9967 | 105.3699 | 1863 | Sweet | 0 | Q1 |
| | | | Xiji County | | | | | | | |
| | L44 | J | Chinese Goji berry | 2020 Jul. 8 | 38.6466 | 106.1531 | 1054 | Mild sweet | 0 | Q3 |
| | L103 | G | Baicaowa Village 1, Wugou Township, Zhenyuan | 2021 Sep. 4 | 35.9093 | 106.8822 | 1531 | Bitter | 10 | Q3 |
| | | | County, Gansu Province | | | | | | | |
| | L17 | N | Daming Dun, Haba Lake, Yanchi | 2019 Nov. 2 | 37.7241 | 107.0733 | 1481 | Bitter | 10 | Q3 |
| | L81 | Q | White Chinese Goji berry frin Golmud Hedong | 2020 Aug. 24 | 36.3974 | 94.9957 | 2809 | Mild sweet | 0 | Q2 |
| | | | Farm, Qinghai | | | | | | | |
| | L21 | N | Yehei 3-1 from north of Shanhe Bridge, Zhongning, | 2019 Oct. 12 | 37.4688 | 105.5401 | 1198 | Mild bitter | 1 | Q2 |
| | | | downstream of Qingshui River | | | | | | | |
| | L46 | N | Lycium ruthenicum (Germplasm Nursery of | 2020 Jul. 8 | 38.6466 | 106.1531 | 1054 | Mild sweet | 0 | Q3 |
| | | | Ningxia Academy of Agricultural and Forestry | | | | | | | |
| | | | Sciences) | | | | | | | |
| | L29 | 0 | Mexican wild Goji berry | 2020 Jul. 8 | 38.5147 | 106.2358 | 1056 | Medium | 7 | Q3 |
| | | | | | | | | bitter | | |
| | L42 | H | Yehong Goji berry No. 2 from Xinxiang, Henan | 2020 Jul. 8 | 38.5147 | 106.2358 | 1056 | Medium | 6 | Q3 |
| | | | | | | | | bitter | | |
| | L40 | N | Yehongku No. 2 from Longde Longzhang Highway | 2020 Jun. 7 | 35.6591 | 106.1058 | 2036 | Bitter | 10 | Q3 |
| | | | Haoshui Gas Station | | | | | | | |
| | L87 | Q | Qingqi No.1 | 2020 Aug. 24 | 36.0482 | 97.5061 | 2965 | Sweet and | 3 | Q3 |
| | | | | | | | | then bitter | | |
| | L71 | M | Honggen Goji berry (vine) from Wulate qianqi, | 2020 Aug. 24 | 40.7359 | 108.6470 | 1023 | Medium | 5 | Q3 |
| | | | Inner Mongolia | | | | | bitter | | |
| | L54 | N | Ningqi No.1 | 2020 Jul. 8 | 38.6466 | 106.1531 | 1054 | Sweet | 0 | Q3 |
| | L1 | N | Yehong No. 1 from east of Zhenhu Road, Xiji | 2019 Sep. 22 | 35.8457 | 105.4641 | 1835 | Medium | 7 | Q3 |
| | | | County | | | | | bitter | | |
| | L24 | N | Yehong from Gaoya Township, Wangtuan, | 2019 Oct. 12 | 36.8314 | 106.0094 | 1355 | Sweet and | 3 | Q3 |
| | | | Tongxin County | | | | | then mild | | |
| | | | | | | | | bitter | | |
| Taxon I | L85 | N | Qingshuihe Goji berry from Matan Village, Xuanhe | 2020 Aug. 24 | 37.4740 | 105.4382 | 1211 | Sweet | 0 | Q3 |
| (subtaxon 2) | | | Town, Shapotou District, Zhongwei City | | | | | | | |
| | L78 | X | Xinjiang Jinghe Black Fruit Turning Red in | 2020 Aug. 24 | 44.5999 | 82.8915 | 320 | Mild | 0 | Q3 |
| | | | Zhongning for 2 Years | | | | | sweet | | |
| | L80 | G | Qixin No.3 fromYinma Farm, Yumen City, Gansu | 2020 Aug. 24 | 40.4455 | 97.0512 | 1403 | Sweet | 0 | Q3 |
| | | | Province | | | | | | | |
| | L97 | G | Ye Hong No. 2 from Long Shou road, Shandan | 2020 Sep. 11 | 38.7196 | 101.1685 | 1828 | Sweet and | 1 | Q3 |
| | | | County, Zhangye City, Gansu Province | | | | | then mild | | |
| | | | | | | | | bitter | | |
| | L95 | G | Yehong, from Sandaogou, Guazhou County, | 2020 Sep. 11 | 40.5202 | 96.7976 | 1384 | Sweet and | 4 | Q3 |
| | | | Jiuquan City, Gansu Province | | | | | then bitter | | |
| | L94 | Q | Yehong No. 4 from Dagele Wulonggou, Dulan | 2020 Sep. 9 | 36.2143 | 95.8734 | 3231 | Sweet and | 2 | Q3 |
| | | | County, Qinghai Province | | | | | then mild | | |
| | | | | | | | | bitter | | |
| | L92 | Q | Yehong No. 2 from Dagele Wulonggou, Dulan | 2020 Sep. 9 | 36.2143 | 95.8734 | 3231 | Sweet and | 2 | Q2 |
| | | | County, Qinghai Province | | | | | then mild | | |
| | | | | | | | | bitter | | |
| | L91 | Q | Yehong No. 1 from Dagele Wulonggou, Dulan | 2020 Sep. 9 | 36.2143 | 95.8734 | 3231 | Sweet and | 2 | Q3 |
| | | | County, Qinghai Province | | | | | then mild | | |
| | | | | | | | | bitter | | |
| | L93 | Q | Yehong No. 3 from Dagele Wulonggou, Dulan | 2020 Sep. 9 | 36.2143 | 95.8734 | 3231 | Sweet and | 4 | Q3 |
| | | | County, Qinghai Province | | | | | then bitter | | |
| | L68 | N | 156-year-old Wild Goji berry (Ancient Tree) from | 2020 Aug. 24 | 36.8891 | 105.7883 | 1532 | Sweet and | 1 | Q3 |
| | | | Huanggu Village, Xinglong, Haiyuan | | | | | then mild | | |
| | | | | | | | | bitter | | |
| | L22 | N | Yehong No. 4 from Shanhe Bridge, Zhongning, | 2019 Oct. 12 | 37.4663 | 105.5421 | 1205 | Sweet | 0 | Q3 |
| | | | downstream of Qingshui River | | | | | | | |
| | L43 | H | Yehong Goji berry No. 3 from Xinxiang, Henan | 2020 Jul. 8 | 38.5147 | 106.2358 | 1056 | [symbol] bitter | 6 | Q3 |
| | L15 | N | Suburb No. 3 from Yanchi County | 2019 Oct. 10 | 37.7844 | 107.4137 | 1340 | Sweet and | 3 | Q3 |
| | | | | | | | | then mild | | |
| | | | | | | | | bitter | | |
| | L16 | N | By Haba Lake in Yanchi County | 2019 Nov. 2 | 37.7079 | 107.0499 | 1456 | Bitter | 10 | Q3 |
| | L14 | N | Suburb No. 2 from South Yanchi County | 2019 Oct. 10 | 37.7848 | 107.4125 | 1335 | Sweet and | 3 | Q3 |
| | | | | | | | | then mild | | |
| | | | | | | | | bitter | | |
| | L13 | N | Old City Wall 1 from Yanchi County | 2019 Oct. 10 | 37.7848 | 107.4125 | 1335 | Sweet and | 3 | Q3 |
| | | | | | | | | then mild | | |
| | | | | | | | | bitter | | |
| | L18 | N | Yehong No. 1 from north Shanhe Bridge, | 2019 Oct. 12 | 37.4652 | 105.5406 | 1200 | Sweet | 0 | Q3 |
| | | | Zhongning, downstream of Qingshui River | | | | | | | |
| Taxon I | L28 | N | Yehong No. 1 from Yanwo Village, Huinong, | 2019 Oct. 16 | 39.0814 | 106.6197 | 1092 | Sweet and | 3 | Q3 |
| (subtaxon 3) | | | Yanzidun, Shizuishan | | | | | then bitter | | |
| | L20 | N | Yehong No. 3 (precocious) from north of Shanhe | 2019 Oct. 12 | 37.4688 | 105.5401 | 1198 | Sweet | 0 | Q3 |
| | | | Briddge, Qingshiui River, Zhongningshan | | | | | | | |
| | L23 | N | Jiaozishan Forest Farm, south of Qingshuihe | 2019 Oct. 12 | 37.4588 | 105.5666 | 1213 | Sweet and | 2 | Q3 |
| | | | Shanhe Bridge, Zhongning | | | | | then [symbol] | | |
| | | | | | | | | bitter | | |
| Taxon II | L89 | M | Inner Mongolia No.4 | 2020 Aug. 24 | 40.7419 | 107.3821 | 1039 | Sweet and | 3 | Q3 |
| | | | | | | | | then bitter | | |
| | L88 | M | Inner Mongolia No.1 | 2020 Aug. 24 | 40.7419 | 107.3821 | 1039 | Sweet and | 3 | Q3 |
| | | | | | | | | then bitter | | |
| | L58 | N | Ningqi No.5 | 2020 Jul. 8 | 38.6466 | 106.1531 | 1054 | Sweet | 0 | Q3 |
| | L63 | N | Ningqi No. 10 | 2020 Jul. 8 | 38.6466 | 106.1531 | 1054 | Sweet | 0 | Q3 |
| | L60 | N | Ningqi No.7 | 2020 Jul. 8 | 38.6466 | 106.1531 | 1054 | Sweet | 0 | Q3 |
| | L47 | N | Lycium truncatum | 2020 Jul. 8 | 38.6466 | 106.1531 | 1054 | Sweet | 0 | Q3 |
| | L55 | N | Ningqi No.2 | 2020 Jul. 8 | 38.6466 | 106.1531 | 1054 | Sweet | 0 | Q3 |
| | L33 | S | Yelv, Sunjia Village Committee, Qinghua Town, | 2019 Nov. 7 | 34.4320 | 107.5356 | 637.5 | Bitter | 10 | Q3 |
| | | | Qishan County, Shaanxi Province | | | | | | | |
| | L69 | N | Goji berry (hemp leaf) from Zhang Weizhong | 2020 Aug. 24 | 37.535 | 105.7231 | 1136 | Sweet and | 1 | Q3 |
| | | | planting, Zhouta Township, Zhongning County, | | | | | then mild | | |
| | | | | | | | | bitter | | |
| | L49 | X | Lycium cylindricum | 2020 Jul. 8 | 38.6466 | 106.1531 | 1054 | Sweet | 0 | Q3 |
| Taxon III | L86 | N | Zhongning Qixin No. 53 | 2020 Aug. 24 | 37.5350 | 105.7231 | 1136 | Sweet | 0 | Q3 |
| | L61 | N | Ningqi No.8 | 2020 Jul. 8 | 38.6466 | 106.1531 | 1054 | Sweet | 0 | Q3 |
| | L70 | N | Qixin 11, Yingpantan Village, Ning’an Town, | 2020 Aug. 24 | 37.5350 | 105.7231 | 1136 | Sweet and | 1 | Q3 |
| | | | Zhongning County | | | | | then mild | | |
| | | | | | | | | bitter | | |
| | L62 | N | Ningnongqi No.9 | 2020 Jul. 8 | 38.6466 | 106.1531 | 1054 | Sweet | 0 | Q3 |
| | L59 | N | Ningqi No.6 | 2020 Jul. 8 | 38.6466 | 106.1531 | 1054 | Sweet | 0 | Q3 |
| | L84 | X | Jinghe No.9 | 2020 Aug. 24 | 44.6035 | 82.8915 | 318 | Sweet | 0 | Q3 |
| | L83 | X | Jinghe No.4 | 2020 Aug. 24 | 44.6035 | 82.8915 | 318 | Sweet and | 2 | Q3 |
| | | | | | | | | then mild | | |
| | | | | | | | | bitter | | |
| | L82 | X | Jinghe No.1 | 2020 Aug. 24 | 44.6035 | 82.8915 | 318 | Sweet | 0 | Q3 |
| | L72 | N | 85 years old, Qingshuihe Zhongning | 2020 Aug. 24 | 37.4663 | 105.5421 | 1205 | Sweet | 0 | Q3 |
| | L30 | N | Yehong No. 3 from Yanwo Village Yanzidun, | 2019 Oct. 16 | 39.0766 | 106.6232 | 1093 | Sweet and | 3 | Q3 |
| | | | Huinong, Shizuishan city | | | | | then mild | | |
| | | | | | | | | bitter | | |
| | L26 | N | Yehong No. 2 from Hongliugou, Mingsha, | 2019 Oct. 17 | 37.5724 | 105.8748 | 1167 | Sweet and | 1 | Q3 |
| | | | Zhongning | | | | | then mild | | |
| | | | | | | | | bitter | | |
| Taxon VI | L51 | Q | Hongzhi Goji berry | 2020 Jul. 8 | 38.6466 | 106.1531 | 1054 | Sweet | 0 | Q3 |
| | L48 | X | Lycium dasystemum | 2020 Jul. 8 | 38.6466 | 106.1531 | 1054 | Sweet | 0 | Q3 |
| | L67 | N | 74-year-old wild Goji berry from Haiyuan County | 2020 Aug. 24 | 36.5666 | 105.6390 | 1829 | Sweet | 0 | Q3 |
| | L76 | M | 62 years old, Erdaoqiao Town, Hangjin houqi, | 2020 Aug. 24 | 40.7567 | 107.0019 | 1041 | Sweet and | 1 | Q3 |
| | | | Bayan Zhuoer City | | | | | then mild | | |
| | | | | | | | | bitter | | |
| | L12 | N | Team 6, Kushui River Flower Temple, Jinyintan, | 2019 Oct. 10 | 37.9291 | 106.2796 | 1098 | Sweet and | 3 | Q3 |
| | | | Wuzhong City | | | | | then mild | | |
| | | | | | | | | bitter | | |
| | L19 | N | Yehong No. 2 from north Shanhe Bridge, | 2019 Oct. 12 | 37.4687 | 105.5406 | 1201 | Sweet | 0 | Q3 |
| | | | downstream of Qingshui River, Zhongning | | | | | | | |
| | L79 | M | 062 (Laoshu), Hangjinhou Banner, Inner Mongolia | 2020 Aug. 24 | 40.8969 | 107.1370 | 1036 | Sweet and | 2 | Q3 |
| | | | Autonomous Region | | | | | then mild | | |
| | | | | | | | | bitter | | |
| | L73 | M | 65 years old, Bayan Zhuoer City, Inner Mongolia | 2020 Aug. 24 | 40.7419 | 107.3821 | 1039 | Sweet | 0 | Q3 |
| | L74 | N | 72 years old(ancient tree), Haiyuan County | 2020 Aug. 24 | 36.5654 | 105.6396 | 1852 | Sweet and | 3 | Q3 |
| | | | QX061:2 | | | | | then mild | | |
| | | | | | | | | bitter | | |
| | L77 | X | 54 years old, Tuoli County, Xinjiang Uygur | 2020 Aug. 24 | 45.9472 | 83.60398 | 1050 | Sweet and | 2 | Q3 |
| | | | Autonomous Region | | | | | then mild | | |
| | | | | | | | | bitter | | |
| | L53 | N | Lycium barbarum var. auranticarpum | 2020 Jul. 8 | 38.6466 | 106.1531 | 1054 | Sweet | 0 | Q3 |
| | L90 | Q | Yehong from Xiwang Street Museum, Dulan | 2020 Sep. 8 | 36.2899 | 98.09399 | 3086 | Sweet and | 4 | Q3 |
| | | | County, Qinghai | | | | | then bitter | | |
| | L75 | M | 103 years old, Bayanzhuoer City, Inner Mongolia | 2020 Aug. 24 | 40.7419 | 107.3821 | 1039 | Sweet and | 2 | Q3 |
| | | | | | | | | then mild | | |
| | | | | | | | | bitter | | |
| | L25 | N | Yehong No.1 Hongliugou, Mingsha, Zhongning | 2019 Oct. 17 | 37.5682 | 105.8786 | 1148 | Sweet and | 1 | Q3 |
| | | | | | | | | then mild | | |
| | | | | | | | | bitter | | |
| Taxon V | L57 | N | Ningqi No. 4 | 2020 Jul. 8 | 38.6466 | 106.1531 | 1054 | Sweet | 0 | Q3 |
| | L56 | N | Ningqi No.3 | 2020 Jul. 8 | 38.6466 | 106.1531 | 1054 | Sweet | 0 | Q3 |
| | L96 | G | Ye Hong No. 1, Long Shou Lu Shandan County, | 2020 Sep. 11 | 38.7151 | 101.1775 | 1840 | Sweet and | 1 | Q3 |
| | | | Zhangye City, Gansu Province | | | | | then mild | | |
| | | | | | | | | bitter | | |

It can be seen that these 110 Goji berry samples may come from three primitive ancestral populations. Among them, 69 samples were from the third original ancestral group; followed by 33 samples from the first original ancestral group; and 8 samples from the second original ancestral group. The samples from these three possible primitive ancestral populations all contained bitter, medium bitter and sweet germplasms. It was shown that “gene exchange” had occurred between these three original ancestral populations.

### (4) Genetic Diversity Analysis

Genetic diversity analysis of 110 Goji berry samples was carried out and the results are shown in Table 5:

TABLE 5
Genetic diversity of 110 Goji berry germplasm populations

| | | Expected_{—} | Expected_{—} | Nei_{—} | Number_{—} | Observed_{—} | Observed_{—} | Polymorphysm_{—} | Shnnon_{—} |
|---|---|---|---|---|---|---|---|---|---|
| | Average_{—} | allele_{—} | heterozygous_{—} | diversity_{—} | of_poly_{—} | allele_{—} | heterozygous_{—} | information_{—} | Wiener_{—} |
| Group | MAF | number | number | index | marker | number | number | content | index |
| Gansu | 0.3035 | 1.5794 | 0.3242 | 0.3455 | 16833 | 1.85786362246458 | 0.0996 | 0.2552 | 0.4764 |
| Henan | 0.4261 | 1.4962 | 0.2603 | 0.3453 | 7830 | 1.56153184165232 | 0.0789 | 0.1991 | 0.3678 |
| Zhonghua | 0 | 0 | 0 | 0 | 1 | 0 | 0 | 0 | 0 |
| Guangdong | 0 | 0 | 0 | 0 | 1 | 0 | 0 | 0 | 0 |
| Inner | 0.1771 | 1.1998 | 0.1288 | 0.1392 | 9818 | 1.50035674243196 | 0.1237 | 0.1077 | 0.2066 |
| Mongolia | | | | | | | | | |
| Ningxia | 0.2248 | 1.5218 | 0.3141 | 0.3171 | 19461 | 1.99179492406483 | 0.1036 | 0.2541 | 0.478 |
| Mexico | 0 | 0 | 0 | 0 | 1 | 0 | 0 | 0 | 0 |
| Korea | 0 | 0 | 0 | 0 | 1 | 0 | 0 | 0 | 0 |
| Qinghai | 0.1732 | 1.2292 | 0.1501 | 0.1619 | 11458 | 1.5839363979207 | 0.1091 | 0.1261 | 0.2416 |
| Shaanxi | 0.2041 | 1.3407 | 0.2185 | 0.2359 | 14324 | 1.73022022838499 | 0.0885 | 0.1814 | 0.3422 |
| Xinjiang | 0.2258 | 1.1692 | 0.1032 | 0.1117 | 6477 | 1.33008867597595 | 0.0892 | 0.0841 | 0.1586 |
| Yunnan | 0 | 0 | 0 | 0 | 1 | 0 | 0 | 0 | 0 |

Note:
Group: population number; Average_MAF: average minor allele frequency; Expected_allele_number: expected number of alleles; Expected_heterozygous_number: expected heterozygosity; Nei_diversity_index: nei diversity index; Number_of_poly_marker: number of polymorphic markers; Observed_allele_number: observed allele number; Observed_heterozygous_number: observed_heterozygosity; Polymorphysm_information_content: Polymorphysm information content (PIC); Shannon_Wiener_index: Shannon Wiener index.

According to Table 5, it can be seen that the genetic diversity of Gansu and Ningxia Goji berry germplasms was strong, with all of the nine parameters: “minor allele frequency”, “expected number of alleles”, “expected heterozygosity”, “nei diversity index”, “number of polymorphic markers”, “number of observed alleles”, “Observed heterozygosity”, “Polymorphism information content (PIC)”, and “Shannon wiener index” being better than those of Goji berry germplasms from other provinces in Northwest China, and therefore, had a strong evolutionary potential. At the same time, it was found that the genetic diversity type of Goji berry germplasms in Ningxia is higher, with 22, 3 and 40 samples from the first, second and third primitive ancestry group, respectively.

The above mentioned is only an example of the present disclosure, not to limit the scope of the patent of the present disclosure. Any equivalent structure or equivalent process transformation made by using the content of the specification of the present disclosure, or directly or indirectly applied in other related technical fields, are included in the scope of patent protection of the present disclosure in the same way.

What is claimed is:

1. A method for determining an evolutionary primitive ancestry of Goji berry, comprising steps of:
digesting DNA of a Goji berry sample with restriction endonucleases RsaI and HinCII, subjecting digested fragments to high-throughput sequencing and bioinformatic analysis to obtain single nucleotide polymorphism (SNP) markers, and determining an evolutionary primitive ancestry of the Goji berry sample by genetic analysis of the SNP markers; wherein
the Goji berry sample includes all species of Chinese Goji berry germplasms of 7 species and 3 varieties, germplasms of Korea in northeast Asia and germplasms of Mexico in America.

2. The method according to claim 1, wherein a tree from which the Goji berry sample comes is from 3 to 156 years old and a sampling site is from 320 m to 3231 m in altitude.

3. The method according to claim 1, wherein he digested fragments have a length of 364-414 bp.

4. The method according to claim 1, wherein the digested fragments are subjected to A-tailing at a 3′ end, ligation with adapters, PCR amplification, purification, mixing, and gel cutting to select target fragments, and high-throughput sequencing is carried out after library quality control.

5. The method according to claim 4, wherein data obtained from the high-throughput sequencing is identified by Dual-index to obtain reads of the Goji berry sample, and after filtration of adaptors for the reads, sequencing quality and data volume are evaluated.

6. The method according to claim 1, wherein bioinformatic analysis comprises acquisition of polymorphic SLAF tags and acquisition of SNP markers.

7. The method according to claim 6, wherein the acquisition of polymorphic SLAF tags comprises clustering reads from sequencing of different Goji berry samples based on sequence similarity.

8. The method according to claim 6, wherein acquisition of SNP markers comprises:
mapping the reads to a reference genome, with a sequence type that has the highest depth in each SLAF tag as a reference sequence, developing SNPs with two methods, GATK and samtools, respectively, and intersecting the SNPs obtained by the two methods to attain SNP markers.

9. The method according to claim 1, wherein the genetic analysis comprises phylogenetic tree analysis, population structure analysis, principal component analysis and linkage disequilibrium analysis.
